(19) United States
(12) Patent Application Publication
(10) Pub. No.: US 2023/0233607 A1
(43) Pub. Date: Jul. 27, 2023

(54) LIGHT-INDUCIBLE GENE ACTIVATION SYSTEMS AND METHODS FOR MAKING AND USING THEM

(71) Applicant: The Regents of the University of California, Oakland, CA (US)

(72) Inventors: Yingxiao WANG, San Diego, CA (US); Ziliang HUANG, San Diego, CA (US); Yiqian WU, San Diego, CA (US)

(21) Appl. No.: 17/800,500

(22) PCT Filed: Feb. 17, 2021
(86) PCT No.: PCT/US2021/018400

§ 371 Date: Aug. 17, 2022

Related U.S. Application Data
(60) Provisional application No. 62/977,569, filed on Feb. 17, 2020.

(51) Int. Cl. A61K 35/17; C12N 15/63; C12N 15/62; C12N 5/0783; C12N 15/90; C12N 15/11; C12N 9/22; C12N 15/86; A61N 5/06; C07K 14/245; C07K 14/47; C07K 14/00; C07K 14/705; C07K 14/725; A61P 35/00; A61K 38/46

(52) U.S. Cl. CPC A61K 35/17; C12N 15/635; C12N 15/625; C12N 5/0636; C12N 15/907; C12N 15/11; C12N 9/22; C12N 15/86; A61N 5/062; C07K 14/245; C07K 14/4728; C07K 14/4702; C07K 14/00; C07K 14/70503; C07K 14/7051; A61P 35/00; A61K 38/465; C07K 14/7056; C12N 2310/20; C12N 2800/80; C12N 2830/002; C12N 2740/15043; A61N 2005/0663; C07K 2319/30; A61K 2039/5156

(57) ABSTRACT

In alternative embodiments, provided are compositions, including recombinant expression systems and vectors, products of manufacture and kits, and methods, for remotely-controlled and non-invasive manipulation of intracellular nucleic acid expression, genetic processes, function and activity in live cells such as T cells in vivo, for example, activating, adding functions or changing or adding specificities for immune cells, for monitoring physiologic processes, for the correction of pathological processes and for the control of therapeutic outcomes. In alternative embodiments, provided are blue-light-mediated light-inducible nuclear translocation and dimerization (LINTAD) systems for gene regulation to control cell activation based on the integration of light-sensitive LOV2-based nuclear localization, light-induced active transportation via the biLINuS motif, and CRY2-CIB1 dimerization that feature high spatiotemporal control to control or alter cell activities in vivo, for example, to limit CAR T cell activity to the tumor site for immunotherapy applications.

### RELATED APPLICATIONS

This Patent Convention Treaty (PCT) International Application claims the benefit of priority under 35 U.S.C. § 119(e) of U.S. Provisional Application Ser. No. (USSN) 62/977,569, filed Feb. 17, 2020. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under HL121365, GM125379, GM126016, CA204704 and CA209629 awarded by the National Institutes of Health (NIH), and CBET1360341 and DMS1361421, awarded by NSF. The government has certain rights in the invention.

### TECHNICAL FIELD

This invention generally relates to improved and focused systems for expressing exogenous nucleic acids in vivo, including expression of anti-cancer chimeric T cell receptors. In alternative embodiments, provided are compositions, including products of manufacture and kits, and methods, for remotely-controlled and non-invasive manipulation of intracellular nucleic acid expression, genetic processes, function and activity in live cells such as T cells in vivo, for example, activating, adding functions or changing or adding specificities for immune cells, for monitoring physiologic processes, for the correction of pathological processes and for the control of therapeutic outcomes. In alternative embodiments, provided are blue-light-mediated light-inducible nuclear translocation and dimerization (LINTAD) systems for gene regulation to control cell activation based on the integration of light-sensitive LOV2-based nuclear localization, light-induced active transportation via the biLINuS motif, and CRY2-CIB1 dimerization that feature high spatiotemporal control to control or alter cell activities in vivo, for example, to limit CAR T cell activity to the tumor site for immunotherapy applications.

### BACKGROUND

Artificial T cell receptors (also known as chimeric T cell receptors, chimeric immunoreceptors, chimeric antigen receptors (CARs)) are engineered receptors, which graft a desired specificity onto an immune effector cell such as a T cell. CAR T cell therapy is becoming a paradigm-shifting therapeutic approach for cancer treatment, particularly with the benefit of resulted central memory T cells capable of lasting for months to years in suppressing the cancer relapse. In this therapy, T cells are removed from a cancer patient and modified to express CARs that target the cancer. These modified T cells, which can recognize and kill the patient's cancer cells, are re-introduced into the patient.

However, major challenges remain before CAR-based immunotherapy can become widely adopted. For instance, the non-specific targeting of the CAR-T cells against normal/nonmalignant tissues (on-target but off-tumor toxicities) can be life-threatening. In fact, off- tumor toxicities against the lung, gray matter in the brain, and cardiac muscles, have caused multiple cases of deaths. While synthetic biology and genetic circuits have been used in attempts to address this issue, there is an urgent need for high-precision control of CAR-T cells to confine the activation in tissue space.

In immunotherapy, the expression of engineered CAR on the cell surface enables T cells to recognize specific antigens on the target cell. This triggers T cell activation and can eventually lead to the elimination of target cells. Clinical trials involving anti-CD19 CAR T cells against B-cell malignancies have shown promising results, demonstrating the therapeutic effects of CAR T cells in cancer treatment. However, the perfusion of constitutively activated CAR T cells into patients may have lethal consequences due to the induced cytokine storm and ‘on-target, off tumor’ toxicity. Therefore, researchers are actively seeking control over the timing and location of the activation of the perfused CAR T cells. Given the complexity of immune system and the largely overlapping functions of its molecular regulators, it is a daunting challenge to manipulate immune system at global levels with predictable net outcomes.

### SUMMARY

In alternative embodiments, provided are methods for remotely-controlling and non-invasively manipulating expression of an exogenous nucleic acid in a cell, or an immune cell, and optionally modifying or adding a target capability or a function to the cell, or immune cell,
wherein optionally the immune cell is a T cell, a primary T cell, a B cell, a monocyte, a macrophage, a dendritic cell or a natural killercell,
wherein optionally the exogenous nucleic acid is contained in a vector or expression cassette,
and optionally the exogenous nucleic acid comprises a nucleic acid encoding (expressing) a protein, and optionally the protein is a therapeutic protein, or a transcriptional or translational regulatory protein, or a receptor, or a recombinant or an artificial T cell receptor (also known as a chimeric T cell receptor, a chimeric immunoreceptor, a chimeric antigen receptor (CAR), an antibody, a single chain antibody, or a single-domain antibody (also known as sdAb or nanobody) or an antibody fragment consisting of a single monomeric variable antibody domain, or a regulator of CRISPR(clustered regularly interspaced short palindromic repeats), such as Cas9 and dCas9, to control endogenous genome and epigenorne regulations, and genome editing
the method comprising:
(a) inserting or expressing in a recombinantly engineered cell, wherein optionally the recombinantly engineered cell is an immune cell or comprises a plurality of cells or immune cells: a blue-light-mediated light-inducible nuclear translocation and dimerization (LINTAD) system as provided herein, for example, as illustrated in FIG. 1A-B, and optionally the LINTAD system comprises:
   (i) a LexA-CIB1-biLINuS or LCB cassette or chimeric nucleic acid comprising: a LexA DNA-binding domain fused to an N-terminus of CIB1 fused to a biLINuS cassette or chimeric nucleic acid comprising a LOV2 domain of an Jα helix and a bipartite light-inducible nuclear localization signal (NLS), wherein the biLINuS is fused to the C-terminus or CIB1,
   (ii) a CRY2-VPR or CV cassette or chimeric nucleic acid comprising: an nuclear location signal peptide (NLS) fused to an N-terminus of CRY2PHR (photolyase homology region of Arabidopsis CRY2, amino acids 1-498) and a strong transcription activator VPR fused to the CRY2PHR C-terminus, and
   (iii) a cassette or chimeric nucleic acid comprising: the exogenous nucleic acid operatively linked to a promoter, optionally a minimal promoter, operatively linked to a LexA binding site (BS), wherein the exogenous nucleic acid is expressed when the LexA BS binds to LexA;
wherein optionally the blue-light-mediated LINTAD system is stably integrated into the genome of the cell; and
(b) exposing the cell to a blue light to drive to activate biLINuS in LCB, thereby unfolding the Jα helix of LOV2 domain and exposing its NLS motif to cause the nuclear translocation of LCB; simultaneously, the CRY2PHR domain in CV is also activated by blue light and can bind the CIB1 domain of LCB with high affinity, and the LCB-CV complex is targeted to the LexA BS on the reporter cassette so that VPR is in close proximity to the minimal promoter, triggering transcription of the exogenous nucleic acid.

In alternative embodiments of methods as provided herein:
the recombinantly engineered cell is administered in vivo, optionally the recombinantly engineered cell is administered to an individual in need thereof in vivo, and optionally the blue light is administered to only a desired area or location in the individual in need thereof, and optionally the desired area or location in the individual in need thereof is a site of a tumor or a growth, and optionally the recombinantly engineered cell is injected into and/or adjacent or approximate to a cancer of a site of a tumor or a growth;
the expressing of the foxed exogenous nucleic acid in the cell adds a function to the cell, or immune cell, or manipulates a physiologic and/or a genetic process in the cell, or immune cell, and optionally when the upregulated nucleic acid is a nucleic acid expressing (encoding) a CAR, a single chain antibody, or a single-domain antibody (also known as sdAb or nanobody) or an antibody fragment consisting of a single monomeric variable antibody domain, or a regulator of CRISPR (clustered regularly interspaced short palindromic repeats), such as Cas9 and dCas9, to control endogenous genome and epigenome regulations, and genome editing, thereby adding a new specificity, function or target cell to a cell, an immune cell or a T cell;
the cell is a human cell or a mammalian cell, or is a recombinantly engineered cell engineered to be transplanted or inserted into a tissue, an organ, an organism or an individual, or is or comprises a non-human transgenic animal genetically engineered to contain one or a plurality of recombinantly engineered cells;
the cell or the individual in need thereof is first exposed to or administered tamoxifen followed by being exposed to or administered a continuous or pulsed blue light,
wherein optionally the cells are exposed to between about 400 to 600 nM 4-OHT, or about 500 nM 4-OHT,
and optionally blue light is applied to the cells between about 2 to 5 hours, or about 3 hours,
and optionally the blue light frequency is about 400 to 500 nM,
and optionally the blue light is applied in a pulsed manner at about 1 second on to about 59 seconds off, or at about 5 seconds on to about 55 seconds off, optionally repeated over a time period of between about 1 hours and 36 hours, or between about 12 hours and 24 hours,
and optionally the blue light is continuously applied to the cells for between about 1 hour and 24 hours, or between about 2 hours and 12 hours;
   a chimeric antigen receptor (CAR) is expressed on a T cell surface after exposure of the T cell to tamoxifen followed by blue light, thereby activating the T cell to attack and/or kill a cancerous tissue, a cancer cell or a tumor cell,
wherein optionally the cancerous tissue, cancer cell or tumor cell is a local or skin or mucosal metastatic head/neck cancer, a melanoma, or a skin cancer or a skin growth;
   the cell is inside the body of an animal or a human in need thereof, and the recombinantly engineered cell is focused on or approximate to a tumor or a dysplastic or dysfunctional tissue; and/or,
   the method is used for the manipulation or correction of a pathological process, optionally, for eradicating a tumor or a cancer in an individual in vivo, wherein optionally the individual is a human or an animal.

In alternative embodiments, sequences of exemplary LINTAD system components as provided herein comprise:

LexA-CIBl-biLINuS
MKALTARQQEVFDLIRDHISQTGMPPTRAEIAQRLGFRSPNAAEEHLKALA
RKGVIEIVSGASRGIRLLQEEEEGLPLVGRVAAGEPASGGGGSGGGGSNGAIG
GDLLLNFPDMSVLERORAHLKYLNPTFDSPLAGFFADSSMITGGEMDSYLSTAG
LNLPMMYGETTVEGDSRLSISPETTLGTGNFKAAKFDTETKDCNEAAKKMTMN
RDDLVEEGEEEKSKITEONNGSTKSIKKMKHKAKKEENNFSNDSSKVTKELEKT
DYIHVRARRGOATDSHSIAERVRREKISERMKFLODLVPGCDKITGKAGMLDEII
NYVOSLOROIEFLSMKLAIVNPRPDFDMDDIFAKEVASTPMTVVPSPEMVLSGYS
HEMVHSGYS SEMVNSGYLHVNPMOOVNTS SGGGGSGGGGSGLATTLERIEKNF
VITDPRLPDNPIIFASDSFLOLTEYSREEILGRNCRFLOGPETDRATVRKIRDAIDN
OTEVTVOLINYTKSGKKFWNLFHLOPMRDOKGDVOYFIGVOLDGTEHVRDAAE
REGVMLIKKTAENIDEAAKRLPDANLAAAAKKKKLD* (SEQ ID NO: 1)

where the LexA domain is bolded, the CIB1 is underlined, and the biLINuS domain is double underlined, noting that the sequence that is neither bolded nor underlined, but italicized, is linker sequence, and

CRY2PHR-VPR
MKMDKKTIVWFRRDLRIEDNPALAAAAHEGSVFPVFIWCPEEEGQFYPGRA
SRWWMKQSLAHLSQSLKALGSDLTLIKTHNTISAILDCIRVTGATKVVFNHL
YDPVSLVRDHTVKEKLVERGISVQSYNGDLLYEPWEIYCEKGKPFTSFNSYW
KKCLDMSIESVMLPPPWRLMPITAAAEAIWACSIEELGLENEAEKPSNALLT
RAWSPGWSNADKLLNEFIEKQLIDYAKNSKKVVGNSTSLLSPYLHFGEISVR
HVFQCARMKQIIWARDKNSEGEESADLFLRGIGLREYSRYICFNFPFTHEQS
LLSHLRFFPWDADVDKFKAWRQGRTGYPLVDAGMRELWATGWMHNRIRV
IVSSFAVKFLLLPWKWGMKYFWDTLLDADLECDILGWQYISGSIPDGHELD
RLDNPALQGAKYDPEGEYIRQWLPELARLPTEWIHHPWDAPLTVLKASGVE
LGTNYAKPIVDIDTARELLAKAISRTREAQIMIGAAPASPKKKRKVEASSPGIR
RLDALISTSLYKKAGYKEASGSGRADALDDFDLDMLGSDALDDFDLDMLGSDA
LDDFDLDMLGSDALDDFDLDMLINSRSSGSPKKKRKVGSOYLPDTDDRHRIEEK
RKRTYETFKSIMKKSPF SGPTDPRPPPRRIAVPSRS S AS VPKPAPOPYPFTS SLSTIN
YDEFPTMVFPSGOISOASALAPAPPOVLPOAPAPAPAPAMVSALAOAPAPVPVLA
PGPPOAVAPPAPKPTOAGEGTLSEALLOLOFDDEDLGALLGNSTDPAVFTDLASV
DNSEFOOLLNOGIPVAPHTTEPMLMEYPEAITRLVTGAORPPDPAPAPLGAPGLP
NGLLSGDEDFSSIADMDFSALLGSGSGSRDSREGMFLPKPEAGSAISDVFEGREV
COPKRIRPFHPPGSPWANRPLPASLAPTPTGPVHEPVGSLTPAPVPOPLDPAPAVT
PEASHLLEDPDEETSOAVKALREMADTVIPOKEEAAICGOMDLSHPPPRGHLDEL
TTTLESMTEDLNLDSPLTPELNEILDTFLNDECLLHAMHISTGLSIFDTSLF* (SEQ
IDNO:2)

where the CRY2PHR domain is bolded, the VPR domain is underlined, noting that the sequence that is neither bolded nor underlined, but italicized, is linker sequence.

In alternative embodiments, provided are uses of a genetically engineered cell as engineered for use in a method as provided herein, as a medicament; or uses of a genetically engineered cell as engineered for use in a method as provided herein, as a medicament in a remotely-controlled and non-invasive manipulation of a physiologic and/or a genetic process in a cell, or an immune cell, or for the addition of a function or a target specificity to the cell, or immune cell, or plurality of cells or immune cells, or for the manipulation or correction of a pathological process, optionally, for eradicating a tumor or a cancer in an individual in vivo.

In alternative embodiments, provided are genetically engineered cells as engineered for use in a method as provided herein for use as a medicament, or for use as a medicament in a remotely-controlled and non-invasive manipulation of a physiologic and/or a genetic process in a cell, or an immune cell, or for the addition of a function or a target specificity to the cell, or immune cell, or plurality of cells or immune cells, or for the manipulation or correction of a pathological process, optionally, for eradicating a tumor or a cancer in an individual in vivo.

In alternative embodiments, provided are kits or formulations comprising a genetically engineered cell as engineered for use in a method as provided herein.

In alternative embodiments, provided are synthetic or chimeric nucleic acids comprising a blue-light-mediated light-inducible nuclear translocation and dimerization (LINTAD) system comprising:
(i) a LexA-CIB1-biLINuS or LCB cassette or chimeric nucleic acid comprising: a LexA DNA-binding domain fused to an N-terminus of CIB1 fused to a biLINuS cassette or chimeric nucleic acid comprising a LOV2 domain of an Jα helix and a bipartite light-inducible nuclear localization signal (NLS), wherein the biLINuS is fused to the C-terminus or CIB1,
(ii) a CRY2-VPR or CV cassette or chimeric nucleic acid comprising: an nuclear location signal peptide (NLS) fused to an N-terminus of CRY2PHR (photolyase homology region of Arabidopsis CRY2, amino acids 1-498) and a strong transcription activator VPR fused to the CRY2PHR C-terminus, and
(iii) a cassette or chimeric nucleic acid comprising: the exogenous nucleic acid operatively linked to a promoter, optionally a minimal promoter, operatively linked to a LexA binding site (BS), wherein the exogenous nucleic acid is expressed when the LexA BS binds to LexA;
wherein optionally the blue-light-mediated LINTAD system is stably integrated into the genome of the cell, and optionally at least one component of the blue-light-mediated LINTAD system comprises SEQ ID NO:1 or SEQ ID NO:2.

In alternative embodiments, provided are kits or formulation comprising a blue-light-mediated LINTAD system as provided herein.

In alternative embodiments, provided are expression vehicles, recombinantly engineered viruses, or vectors comprising or having contained therein at least one component of a blue-light-mediated LINTAD system as provided herein, wherein optionally the recombinantly engineered virus is a lentivirus, and optionally the at least one component of a blue-light-mediated LINTAD system comprises SEQ ID NO:1 or SEQ ID NO:2.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-B schematically illustrates exemplary an light-inducible nuclear translocation and dimerization (LINTAD) gene activation system as provided herein:

FIG. 1A schematically illustrates the three components of the LINTAD system. LexA-CIB1-biLINuS (LCB): LexA fused with CIB1 and the bipartite light-inducible nuclear localization signal (biLINuS); CRY2PHR-VPR (CV): CRY2PHR fused with VPR; Light-inducible reporter: LexA binding sequence (LexA BS) fused with a minimal promoter and a target gene; also illustrating that in the dark state, LCB stays in the cytoplasm and CV in the nucleus; and

FIG. 1B schematically illustrates how upon blue light stimulation, the Jα helix of LOV2 domain in biLINuS is unfolded to expose the NLS peptide (upper part FIG. 1B), which leads to LCB translocation into the nucleus ((red) dashed line 1 in FIG. 1A); and LexA then binds to LexA BS on the light-inducible reporter ((red) dashed line 2 in FIG. 1A; and meanwhile, CRY2PHR binds to CIB1 upon blue light stimulation ((red) dashed line 3 in FIG. 1A, thus targeting VPR to the minimal promoter region to trigger the reporter gene expression;

as discussed in further detail in Example 1, below.

FIG. 2A-E illustrates characterization of the exemplary LINTAD gene activation in HEK 293T cells:

FIG. 2A illustrates images of HEK 293T cells transfected with LexA-CIB1-mCherry-biLINuS (LCmB) to track the nuclear localization of LCB before and after light stimulation;

FIG. 2A illustrates images of Light-inducible mNeonGreen expression in HEK 293T cells co-transfected with LCB, CV and the light-inducible mNeonGreen reporter;

FIG. 2C graphically illustrates data where: Dark, without light stimulation; and, Light, with 24 hr light stimulation, comparison of mNeonGreen (mNG) intensity of cells transfected with LINTAD and mNeonGreen reporter with (Light) or without light stimulation (Dark);

FIG. 2D graphically illustrates data comparison of light-inducible systems with or without the light-inducible nuclear localization signal (biLINuS). HEK 293T cells were co-transfected with LexA-CIB1-biLINuS (With biLINuS) or LexA-CM1 (No biLINuS), CV, the light-inducible firefly luciferase reporter, and a constitutive Renilla luciferase as an internal reference to normalize the induced firefly luciferase expression in each group;

FIG. 2E schematically (left image) and graphically (right) illustrates comparison of CD47 binding of cells transfected with LCB, CV and the light-inducible CV1 reporter with or without light stimulation; Left: schematics of CV1-CD47 binding assay; Right: representative flow cytometry histograms of PE staining (Streptavidin-PE) of CD47 under different conditions;

as discussed in further detail in Example 1, below.

FIG. 3A-F illustrates how an exemplary LINTAD system can induce target gene expression upon light stimulation in Jurkat cells:

FIG. 3A graphically illustrates characterization of LINTAD system with light-inducible firefly luciferase (Flue) reporter; Jurkat cells were transfected with (i) both LCB and CV components (LINTAD) or (ii) only the LCB component (LCB only) or (iii) only the CV component (CV only), and all groups were also co-transfected with the light-inducible Fluc reporter, and fluc activities were measured using the same number of cells in each group;

FIG. 3B graphically illustrates representative flow cytometry charts showing light-inducible CD19CAR expression in Jurkat cells transfected with LINTAD regulators LCB and CV, and the light-inducible CD19CAR reporter; the whole live cell population is shown in each chart; CAR expression was quantified by staining of the Myc tag fused to the extracellular domain of CD19CAR;

FIG. 3C graphically illustrates comparison of CAR+cell percentage of the Dark and Light groups shown in FIG. 3B;

FIG. 3D graphically illustrates representative flow cytometry charts showing CD69 levels of the Light and Dark groups, where Jurkat cells were transfected with LINTAD regulators and the CD19CAR-YPet reporter, co-cultured with CD19-expressing Toledo cells after light/dark treatment, and stained with anti-CD69 antibody for flow cytometry analysis;

FIG. 3E graphically illustrates comparison of CD69+cell percentage of the Dark and Light groups shown in FIG. 3D;

FIG. 3F graphically illustrates representative CD69 expression profiles of Jurkat cells hosting light-inducible headless CAR reporter after co-culture with CD19-expressing target cells and anti-CD69 antibody staining; the YPet+populations were used for CD69 comparison,

as discussed in further detail in Example 1, below.

FIG. 4A-G graphically and schematically illustrate how light-inducible cytotoxicity of engineered primary human T cells in vitro and light-inducible gene activation in vivo:

FIG. 4A graphically illustrates characterization of LINTAD-mediated Fluc induction in primary human T cells, where T cells infected with LINTAD regulators and Fluc reporter (with constitutive Rluc as internal reference) were (i) kept in dark (Dark) or stimulated by blue light for (ii) 6 hr, (iii) 12 hr, (iv) 24 hr, and the gene induction level was represented by the ratio of Fluc to Rluc luminescence in each group, and normalized to the mean value of the Dark group;

FIG. 4B graphically illustrates in vitro light-inducible cytotoxicity of T cells with CD19CAR reporter, where engineered T cells were either without light stimulation (Dark) or stimulated with blue light for 6 hr or 12 hr, followed by co-culture with Fluc+Nalm-6 tumor cells for 24 hr, and uninfected T cells (UIF) were used as control following the same procedures;

FIG. 4C-D graphically illustrate cytokines IL-2 (FIG. 4C) and IFN-γ (FIG. 4D) secretion from T cells engineered with LINTAD and CD19CAR reporter after co-culture with Nalm-6 cells quantified by ELISA; Dark, without light stimulation. Light, with 12 hr blue light stimulation;

FIG. 4E schematically illustrates an exemplary mouse model used for in vivo light-inducible gene activation, where engineered light-inducible Nalm-6 cells infected with LINTAD and Fluc reporter (with constitutive Rluc as internal reference) were subcutaneously injected into both flanks of NSG mice;

FIG. 4F illustrates representative bioluminescence images showing the induced Fluc expression by blue light (at the region highlighted by the arrow);

FIG. 4G graphically illustrates statistical comparison of light-inducible Fluc activation in mice, where induction level was indicated by Fluc/Rluc ratio at the same site. All Fluc/Rluc values were normalized to that of “Before, Dark Side”, as discussed in further detail in Example 1, below.

FIG. 5A-F graphically and schematically illustrate in vivo cytotoxicity of light-activated primary human T cells:

FIG. 5A graphically and schematically illustrates gene induction efficiency of LINTAD strongly depends on concentrations of regulators, where primary human T cells were infected with LCB-2A-tBFP and eGFP-2A-CV to indicate the expression levels of LINTAD regulators, and light-inducible mCherry reporter was used to determine the gene induction level;

FIG. 5B graphically illustrates light-inducible gene expression using WW-LINTAD in primary T cells; where Light and Dark: T cells infected with exemplary lentiviral WW-LINTAD (LWWCB and WP1CV) and Fluc reporter (with constitutive Rluc as internal reference) were stimulated with (Light) or without (Dark) for 12 hr. CTL: control T cells infected with only the LWWCB component and reporter;

FIG. 5C graphically illustrates cytotoxicity of T cells engineered with exemplary WW-LINTAD, light-inducible Cre reporter and loxP-ZsGreen-stop-loxP-CD19CAR (see FIG. S9; where UIF: uninfected T cells, CTL: control T cells infected with only the LWWCB component of exemplary WW-LINTAD, Cre reporter, and loxP-ZsGreen-stop-loxP-CD19CAR; and Dark: without light stimulation; Light: with 12 hr light stimulation;

FIG. 5D-F graphically and in images illustrate in vivo cytotoxicity of light-inducible T cells after in vitro light stimulation: NSG mice were subcutaneously injected with Fluc+Nalm-6 cells on the right flank; primary human T cells engineered with exemplary WW-LINTAD, light-inducible Cre reporter and loxP-ZsGreen-stop-loxP-CD19CAR were treated with (Light) or without (Dark) 12 hr blue light, followed by local injection into the mice at the tumor sites and uninfected T cells (“UIF”) were used as control following the same procedures;

FIG. 5D schematically illustrates a timeline of tumor inoculation and T cell injection;

FIG. 5E illustrates images of tumor burden as quantified by bioluminescence imaging (BLI) after tumor inoculation for 16 days;

FIG. 5F graphically illustrates quantification of tumor aggressiveness in different groups in FIG. 5E, where the integrated luminescence of a tumor at each time point was normalized to that of the same tumor on Day 3,

as discussed in further detail in Example 1, below.

FIG. 6A-D graphically and in images illustrate that in vivo light stimulation of exemplary LINTAD CAR T cells can control cytotoxicity with high spatial resolution:

FIG. 6A schematically illustrates an exemplary mouse model and experiment timeline, where Fluc+Nalm-6 cells were subcutaneously injected on both flanks of the mouse, and exemplary LINTAD CAR T cells were locally injected into the inoculated tumor sites 4 days later;

FIG. 6B illustrates bioluminescence images of mice in the experiment described in FIG. 6A;

FIG. 6C graphically illustrates quantification of tumor growth in FIG. 6B;

FIG. 6D graphically illustrates the same experiment procedures as in FIG. 6A with uninfected T cells instead of LINTAD CAR T cells,

as discussed in further detail in Example 1, below.

FIG. 7A-E (also called “FIG. S1”) illustrate images of nuclear translocation of the exemplary cassette LexA-CIB1-mCherry-biLINuS showing that it is specific to blue light stimulation;

FIG. 7A illustrates fluorescence images of HEK 293T cells expressing the exemplary cassette LexA-CIB1-mCherry-biLINuS (LCmB) before and after 10 min of 560 nm light stimulation (0.5 s/30 s);

FIG. 7B illustrates fluorescence images of the same cells in FIG. 7A before and after 10 min of 460 nm light exposure;

FIG. 7C illustrates a representative image showing the nuclear localization of EGFP-CV in HEK 293T cells;

FIG. 7D illustrates fluorescence images of HEK 293T cells expressing eGFP with (Light) or without (Dark) 24 hr light stimulation;

FIG. 7A illustrates fluorescence images of HEK 293T cells expressing tBFP with (Light) or without (Dark) 24 hr light stimulation,

as discussed in further detail in Example 1, below.

FIG. 8A-C (also called “FIG. S2”) illustrate images that characterize LINTAD-mediated gene induction in HEK 293T cells using an exemplary dual-luciferase reporter system;

FIG. 8A schematically illustrates exemplary DNA cassettes used in the dual-luciferase reporter system: in the upper image, LexA BS: LexA-binding DNA sequence; Fluc, firefly luciferase gene; and in the lower image, TK, constitutive HSV-thymidine kinase promoter; Rluc, Renilla luciferase gene; stop codon after each gene is indicated;

FIG. 8B graphically illustrates light-inducible gene activation using an exemplary LINTAD in HEK 293T cells, where cells were transfected with intact LINTAD system (LCB and CV, shown as “LINTAD” in figure) or LCB (“LCB only”) or CV (“CV only”), together with the dual-luciferase reporter constructs shown in FIG. 8A;

FIG. 8B graphically illustrates comparison of exemplary LINTAD systems using CRY2PHR and CRY2low in HEK 293T cells; for the original LINTAD group, cells were co-transfected with LCB and CV-expressing plasmids; for the LINTAD (CRY2low) group, cells were transfected with LCB and CRY2low-VPR; and, both groups were also transfected with the light-inducible firefly luciferase reporter and the constitutive Renilla luciferase-expressing plasmid as internal control,

as discussed in further detail in Example 1, below.

FIG. 9A-F (also called “FIG. S3”) graphically illustrate data showing exemplary LINTAD-mediated light-inducible gene expression in Jurkat cells, where the Jurkat cells were transfected with the exemplary LINTAD system (LCB and CV) with different light-inducible reporter cassettes (mNeonGreen reporter in FIG. 9A and FIG. 9C; CD19CAR-YPet in FIG. 9B and FIG. 9D), and cells were stimulated with blue light for 24 hr (Light) or kept in dark for 24 hr (Dark) before fluorescence measurement:

FIG. 9A-B graphically illustrate comparison of mean fluorescent intensity;

FIG. 9C-D graphically illustrate representative fluorescence profiles of light-induced or non-induced cells shown in FIG. 9A or FIG. 9B, the YPet+ gate was indicated with dotted line in FIG. 9D;

FIG. 9E graphically illustrates dynamics of CD19CAR expression after light induction in Jurkat cells, where Light stimulation was applied from day 1 to day 2 for 24 hr as indicated by the bar below the x-axis. CD19CAR expression was measured by myc-tag staining;

FIG. 9F graphically illustrates representative expression (YPet fluorescence) profiles of Jurkat cells transfected with LCB, CV and the light-inducible YPet-tagged headless CAR (without CD19 recognition domain) reporter with (Light) or without (Dark) 24 hr light stimulation, as discussed in further detail in Example 1, below.

FIG. 10A-F (also called “FIG. S4”) schematically illustrate exemplary lentiviral vectors used for T cell infection;

FIG. 10A schematically illustrates the exemplary constitutive LCB lentiviral vector containing a PGK promoter followed by: LCB gene, WPRE element, flanked by 5′LTR and 3′LTR;

FIG. 10B schematically illustrates the exemplary constitutive CV viral vector has the same design as LCB lentiviral vector, except containing the CV gene instead of LCB;

FIG. 10C schematically illustrates the exemplary light-inducible Fluc reporter lentiviral vector contains the LexA binding sequence (LexA BS), followed by minimal promoter, Fluc gene, PGK promoter, and Rluc gene (for Fluc normalization);

FIG. 10D schematically illustrates the exemplary light-inducible CD19CAR reporter lentiviral vector contains LexA BS, minimal promoter and Myc-tagged CD19CAR gene and WPRE element, flanked by 5′LTR and 3′LTR;

FIG. 10E graphically illustrates infection efficiency of an exemplary LINTAD system in primary T cells; where primary T cells were co-infected with the exemplary lentiviruses encoding LCB-P2A-tBFP, EGFP-P2A-CV, and LIP-myc-CD19CAR-PGK-mCherry; Left image shows the mCherry expression profile in the infected T cells; and right image shows the EGFP-tBFP expression profile of mCherry+cells, as discussed in further detail in Example 1, below.

FIG. 11A-C (also called “FIG. S5”) illustrate images of an exemplary light stimulation system used for in vivo studies:

FIG. 11A illustrates an image overview of the light stimulation system containing home-built LED control box with intensity knobs (for two LEDs), on/off timer (for controlling on/off cycle pattern of LEDs), and blue-light emitting LED; and each item was indicated in the figure;

FIG. 11B illustrates an image of a representative mouse with a blue LED attached on the desired stimulation region;

FIG. 11B illustrates an image of an exemplary LED control box with intensity knobs; the blue LED used in FIG. 11B is also shown here (wavelength 460 nm, diameter 3 mm), as discussed in further detail in Example 1, below.

FIG. 12A-C (also called “FIG. S6”) illustrate images showing that the level of LINTAD gene activation depends on the expression level of regulators:

FIG. 12A-B graphically illustrate HEK 293T cells transfected with eGFP-2A-CV, and LCB-2A-tBFP and CD19CAR reporter cassette were stimulated with light (“Light”) or kept in dark (“Dark”) for 24 hr before flow cytometry measurement;

FIG. 12C graphically illustrates three-dimensional plots showing the relationship between gene induction and regulator expression levels, where human primary T cells were infected with viral vectors expressing LCB-2A-tBFP and eGFP-2A-CV (or eGFP-2A-CVP64 using VP64 instead of VPR as the transcriptional activator as indicated in figure) and the viral vector of light-inducible mCherry reporter, as discussed in further detail in Example 1, below.

FIG. 13A-B (also called “FIG. S7”) illustrate images showing engineering of exemplary LINTAD expression systems with weak dimer helpers:

FIG. 13A schematically illustrates exemplary construct designs of LINTAD with various weak dimer helpers: LINTAD, original LINTAD system without weak dimer helper; G1-10, split super folder GFP1-10 fragment; G11, split super folder GFP11 fragment;

FIG. 13B graphically illustrates comparison of light-induction capability of the exemplary LINTAD expression systems shown in FIG. 13A at different regulator expression levels, as discussed in further detail in Example 1, below.

FIG. 14A-C (also called “FIG. S8”) graphically illustrate images of exemplary LINTAD expression systems with weak dimer helpers in T cells;

FIG. 14A graphically illustrates where primary human T cells were infected with viral vectors expressing WW-2 (indicated as “WW” in figure) or SH3-2 (indicated as “SH3” in figure, see FIG. 13A) and viral vector of dual-luciferase reporter;

FIG. 14B-C graphically illustrate in vivo light stimulation of LINTAD CAR T cells can control cytotoxicity with high spatial resolution (plots showing individual data points for FIG. 6C-D);

FIG. 14B illustrates data where exemplary LINTAD CAR T cells were subcutaneously injected into two sites of the same mice (left and right flanks), where tumor cells (Nalm-6 with constitutive Fluc) had been inoculated 4 days before; left side was illuminated with blue light for 12 hr (light); right side was covered with foil (dark);

FIG. 14B illustrates data of same experiment procedures as in FIG. 14B, but uninfected T cells were used instead of LINTAD CAR T cells, tumor size was measured by Fluc luminescence reading, as discussed in further detail in Example 1, below.

FIG. 15A-B (also called “FIG. S9”) schematically illustrate the design of exemplary lentiviral vectors used for in vivo cytotoxicity studies:

FIG. 15A illustrates schematics of light-inducible Cre reporter and loxP-ZsGreen-STOP-loxP-CD19CAR cassettes in the cells (LINTAD regulators not shown); once LINTAD system in the cell is activated by light, Cre can be induced to express and catalyze recombination between loxP sites on the loxP-ZsGreen-STOP-loxP-CD19CAR cassette, thus switching on constitutive CD19CAR expression in the cells;

FIG. 15B illustrates exemplary lentiviral vectors for expression of WW-LINTAD (WW-2 in FIG. 13A) and light-inducible Cre and loxP-ZsGreen-STOP-loxP-CD19CAR (before and after recombination) cassette, as discussed in further detail in Example 1, below.

Like reference symbols in the various drawings indicate like elements.

### DETAILED DESCRIPTION

In alternative embodiments, provided are compositions, including products of manufacture and kits, and methods, for remotely-controlled and non-invasive manipulation of physiologic or genetic processes and/or protein expression in live cells in vivo, for example, immune cells such as T cells, for example, the controlled expression of recombinant nucleic acids or proteins such as for example, chimeric T cell receptors, chimeric immunoreceptors or chimeric antigen receptors (CARs), for the manipulation of physiologic processes in the cell or for the correction of pathological processes (for example, non-specific targeting of the CAR-T cells against normal/nonmalignant tissues) and/or for control of therapeutic outcomes, for example, engineered T cells expressing CARs targeting specific cancers cells and killing them.

In alternative embodiments, provided are compositions and methods for the manipulation or correction of pathological processes, for example, for eradicating tumors and cancers in human subjects, without limitation in penetration depth of an inducible signal, that comprise use of blue-light-mediated light-inducible nuclear translocation and dimerization (LINTAD) systems for gene regulation to control cell activation based on the integration of light-sensitive LOV2-based nuclear localization, light-induced active transportation via the biLINuS motif, and CRY2-CIB1 dimerization. In alternative embodiment, provided are compositions and methods for inducing expression of nucleic acids, for example, genes, in immune cells such as T cells, monocytes/macrophages, dendritic cells, natural killercells and the like. In alternative embodiment, provided are compositions and methods for stimulating or inhibiting ligand-receptor interactions, including any surface molecular interaction, including but not limiting to inhibitory CTLA-4 and apoptotic Fas.

In alternative embodiments, provided are compositions and methods for the treatment, amelioration, prevention or eradication of a pathologic process or a pathology, a disease, an abnormal tissue, or an infection, for example, bacterial or viral infections, with a specific cell surface marker. In alternative embodiment, provided are compositions and methods for the controlled production of RNAs (including microRNA, long non-coding RNAs), and for the epigenetic and genetic modulation of molecules for the treatment, amelioration, prevention or eradication of a pathologic process, a disease, an abnormal tissue, or an infection.

In alternative embodiments, provided are engineered cells, for example, human cells, for example, immune cells, for example, T cells, capable of inducibly expressing a nucleic acid, for example, a protein encoding nucleic acid, for example, expressing a recombinant protein such as a chimeric antigen T cell receptor (CAR), by operatively linking a gene of interest, i.e., a gene (for example, a gene expressing a CAR), to a blue-light-mediated light-inducible nuclear translocation and dimerization (LINTAD) system for manipulating gene expression.

In alternative embodiments, provided are engineered light-controllable cells, for example, T cells, for example, CAR T cells, for example, for cancer immunotherapy. Engineered chimeric antigen receptor (CAR) T cells as provided herein can detect and eradicate cancer cells within patients, and provides truly cancer-specific CAR-targeting cell surface antigens to prevent potentially fatal on-target off-tumor toxicity against other healthy tissues within the body. T cells engineered to express chimeric antigen receptors (CARs) on cell surface as provided herein can recognize and engage with target cancer cells with redirected specificity. This CAR T cell-mediated immunotherapy can be a therapy for cancer that reduces adverse effects such as on-target off-tumor cytotoxicity, cytokine release syndrome and tumor lysis syndrome. In alternative embodiments, provided are engineered controllable CAR T cells that achieve high-precision control over their activation. Optogenetics utilizing genetically encoded dimerizers has been applied to remotely activate gene expression. In alternative embodiments, provided are blue-light-mediated light-inducible nuclear translocation and dimerization (LINTAD) systems for gene regulation to control cell activation based on the integration of light-sensitive LOV2-based nuclear localization, light-induced active transportation via the biLINuS motif, and CRY2-CIB1 dimerization.

We demonstrated light-controllable gene expression and functional modulation in HEK 293T and Jurkat T cell lines. We further improved and applied the LINTAD system in primary human CAR T cells and showed that the light-stimulated cells possessed a significantly stronger cytotoxicity against target cancer cells than the non-stimulated ones, both in vitro and in vivo. Therefore, our newly developed LINTAD system can serve as an efficient and general tool to remotely and non-invasively control gene activation in live cells for therapeutic applications.

We have developed a light-inducible, blue-light-mediated, gene activation system LINTAD by integrating the LOV2-based light-inducible nuclear localization, light-induced active transportation via the biLINuS motif, and CRY2-CIB1 dimerization. While the CRY2-CIB1 dimerization has been utilized to engineer various gene activation systems, the basal dimerization tendency of the CRY2-CIB1 pair can result in a relatively high level of leaky expression of reporter genes^{1,2}. The blue-light-mediated LINTAD system described herein utilizes both the light-induced active transportation via the biLINuS motif and the pulling effect mediated by the CRY2-CIB1 dimerization for nuclear translocation to control gene activation. Without light, CIB1 stays outside of the nucleus and is decoupled from CRY2 to achieve a low basal leakage of target gene expression.

The modular design of exemplary LINTAD systems as provided herein allow a simple change of the DNA binding domain, the dimerization pair, and/or the transcription activator to integrate different molecular interactions for the control of desired protein production and functional cellular outcomes. Replacing the reporter gene with different target genes can conveniently switch, in principle, any functional outcome of LINTAD monitor or reprogram host cells^{1,3-5}. For example, TRAIL (TNF-related apoptosis inducing ligand), an inducer of apoptosis and a cancer therapeutic^{6,7}, can be used as the target reporter gene and LINTAD can be utilized to achieve light-inducible tumor suppression/killing.

In alternative embodiments, the reporter genes are regulators of CRISPR (clustered regularly interspaced short palindromic repeats), such as Cas9 and dCas9. Exemplary light and LINTAD systems as provided herein hence can be applied to control genome editing with high precision in space and time.

In alternative embodiments, the DNA binding domain LexA is replaced by other types of its kind (with the corresponding binding sequence on the reporter) to orthogonally target and drive different gene products^{1,2,8}, and expression levels can also be fine-tuned through promoter sequence engineering⁹. The VPR transcription activator can be switched to smaller domains such as VP64 to reduce the gene cargo size for enhanced delivery efficiency in primary cells which are usually difficult to transfect or infect^{4,10}.

Cell-based immunotherapies such as CAR T cells are becoming a paradigm-shifting therapeutic approach with promising success in treating blood tumors, despite drawbacks such as specificity and cytokine release syndrome¹¹⁻¹⁴. With the next frontier of applying cell-based therapies in solid tumors, one critical component of technology development is to gain spatiotemporal control of engineered T cells with high precision^{15,16}. LINTAD can precisely address this need, particularly for tumors at shallow surfaces, including skin cancers and melanoma. Integrated with optical fibers, LINTAD can also be extended to control engineered T cells targeted at gastrointestinal tracts to treat gastrointestinal cancers¹⁷. As such, LINTAD could provide high precision and power of controlling engineered cells for cell-based therapeutics.

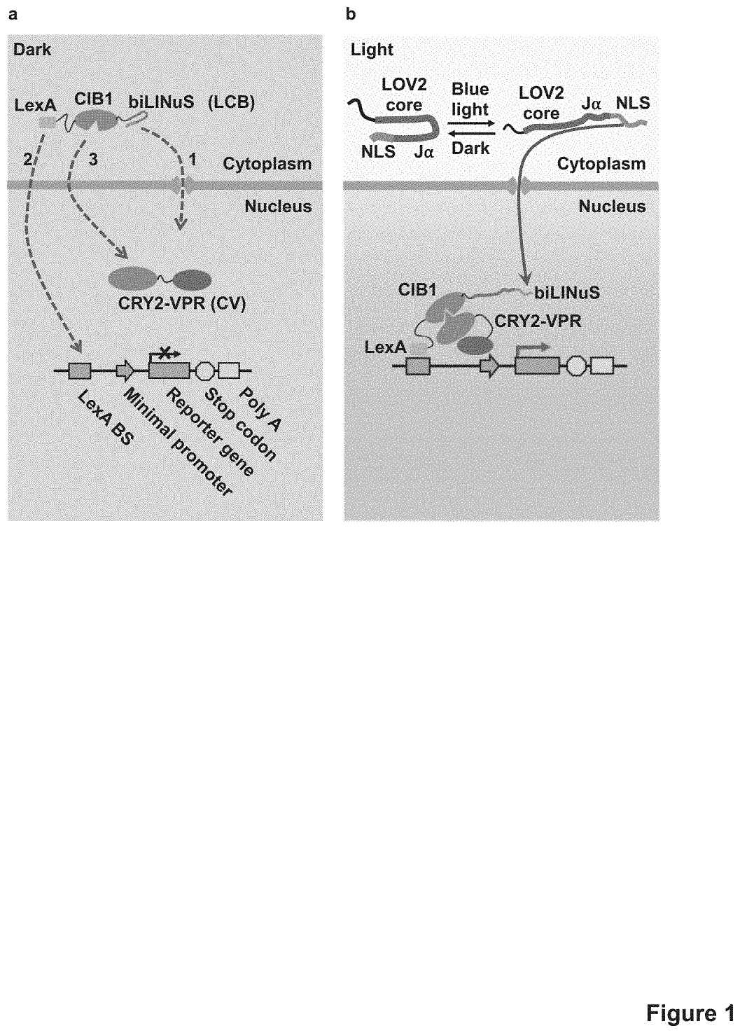

In summary, we have engineered and characterized a new blue-light-mediated light-controllable modular system, or blue-light-mediated LINTAD system, for gene activation and cancer immunotherapy, with a precise control in space and time and hence efficiency and safety. We fused the LexA DNA-binding domain^{18,19} to the N-terminus of CIB⁴ and a bipartite light-inducible nuclear localization signal (biLINuS¹⁹) to its C-terminus (LexA-CIB1-biLINuS or LCB, FIG. 1a).

Meanwhile, we fused nuclear localization signal (NLS) to the N-terminus of CRY2PHR (photolyase homology region of Arabidopsis CRY2, amino acids 1498)^{2,3} and a strong transcription activator VPR¹° to its C-terminus (CRY2-VPR or CV). Light-inducible reporters were constructed by assembling the LexA binding sequence (LexA BS), the minimal promoter, and the target genes. In the dark state, LCB stays in the cytoplasm while CV stays in the nucleus, thus spatially separated in different cellular compartments (FIG. 1a). Upon blue light stimulation, the biLINuS in LCB is activated, unfolding the Jα helix of LOV2 domain and exposing its NLS motif¹⁹ to cause the nuclear translocation of LCB (FIG. 1b). Simultaneously, the CRY2PHR domain in CV is also activated by blue light and can bind the CIB1 domain of LCB with high affinity^{1,2,4}. The LCB-CV complex is targeted to the LexA BS on the reporter cassette so that VPR is in close proximity to the minimal promoter, triggering transcription of the target gene (FIG. 1b). This “push-pull” strategy incorporating the biLINuS-enabled active nuclear translocation of LCB as well as the pulling effect of the CIB1-CRY2 dimerization should lead to strong gene activation upon blue light stimulation.

### CRISPR Systems for Genome Integration

In alternative embodiments, a CRISPR method is one alternate method of engineering the target cells (for example, for inserting the exemplary light-inducible nuclear translocation and dimerization (LINTAD) systems as provided herein in target cells such as T cells), for example, as an alternative to using the exemplary lentivirus infection method also described herein.

For example, in alternative embodiments, a SpCas9 or equivalent protein and a guide RNA are expressed or delivered into a target cell, for example, through electroporation or equivalents. The CAR reporter as provided herein is included in a template plasmid which contains nucleic acid sequence homologous to the targeted insertion region of target cell genome, and is delivered into target cells, for example, through electroporation or equivalents. After CRISPR-mediated homology-directed repair, the CAR reporter is inserted in the target cell genome. Successful integration of the CAR reporter into the target cell genome can be verified by genotyping methods and/or function-based assays.

Regardless of which exemplary delivery method is used (for example, lentivirus or CRISPR), in alternative embodiments the system is still an exemplary light-inducible nuclear translocation and dimerization (LINTAD) systems as provided herein.

In alternative embodiments, for using CRISPR (for example, instead of the exemplary lentivirus) to engineer target cells, the components needed are: (1) SpCas9 protein (either protein form or nucleic acid form which can express SpCas9 protein when in cells); (2) a single guide RNA (sgRNA, either RNA form or nucleic acid form which can be transcribed into RNA when in cells); and (3) a template plasmid which contains the CAR reporter (including the exemplary Cre-loxP structure), flanked by nucleic acid sequence homologous to the targeted insertion locus/loci (determined by sgRNA) of genome of the cell. With all these components, the cells can go through CRISPR-mediated homology-directed repair, which can insert the CAR reporter in the genome.

In alternative embodiments, any CRISPR system can be used to practice embodiments as provided herein, for example, as described in U.S. Pat. Nos: 10,767,168; 10,760,081; 10,745,716; 10,711,285; 10,711,284; 10,668,173; and/or

### Products of Manufacture and Kits

Provided are products of manufacture and kits for practicing methods as provided herein, including for example exemplary expression cassettes as provided herein, including for example, exemplary light-inducible nuclear translocation and dimerization (LINTAD) systems as provided herein, and including for example cell comprising or having contained therein an exemplary LINTAD system as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of may be replaced with either of” the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims. The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor

Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press

(UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR - Basics: From Background to Bench, First Edition, Springer Verlag, Germany.

### Example 1: Engineering Light-controllable CAR T Cells for Cancer Immunotherapy

This example demonstrates that methods and recombinantly engineered cells as provided herein including the exemplary light-inducible nuclear translocation and dimerization (LINTAD) system for gene regulation are effective and can be used to treat cancer and tumors.

T cells engineered to express chimeric antigen receptors (CARs) on cell surface can recognize and engage with target cancer cells with redirected specificity for cancer immunotherapy. However, there is a lack of ideal CARs for solid tumor antigens which may lead to severe adverse effects, including on-target off-tumor cytotoxicity and cytokine release syndrome. One of the solutions is to engineer CAR T cells to achieve on-off control at confined tissue regions with high spatiotemporal precisions. Optogenetics utilizing genetically encoded dimerizers can precisely control gene activities in space and time. Here, we developed a new light-inducible nuclear translocation and dimerization (LINTAD) system for gene regulation to control CAR T activation by light, combining light-sensitive LOV2-based nuclear localization and CRY2-CIB1 dimerization. We first demonstrated light-controllable gene expression and functional modulation in HEK 293T and Jurkat T cell lines. We then improved the

LINTAD system to achieve optimal light-activatable efficiency at low copy numbers of genetic cassettes to be compatible for gene expression in primary human T cells. The results showed that pulsed light stimulations can activate light-inducible LINTAD CAR T cells with strong cytotoxicity against target cancer cells, both in vitro and in vivo. Therefore, our newly developed LINTAD system can serve as an efficient and general tool to non-invasively control gene activation and activate inducible CAR T cells for precision cancer immunotherapy.

Adoptive cell transfer using patient-derived T cells engineered ex vivo with chimeric antigen receptors (CARs) has emerged as a promising therapeutic strategy for cancer treatment (1, 2). Upon antigen engagement, CAR T cells can initiate anti-tumor cytokine production and target tumor cell killing (3). Meanwhile, CAR T cells can be stimulated to proliferate exponentially, resulting in a highly amplified T cell response and elimination of tumor cells within weeks (4). However, extensive research has revealed the limitations of CAR T cell-based immunotherapy (5, 6). First, CAR T cells may recognize and engage with normal cells expressing target antigens even at low levels (7, 8). This on-target off-tumor toxicity is especially lethal when the infused CAR T cells are activated by the normal cells expressing low levels of target antigens in the heart, liver or lung, potentially leading to death (9). Second, even with specific tumor targeting, cytokine release syndrome and tumor lysis syndrome can occur following a large dosage of CART cell infusion (10). Furthermore, it remains a challenge to predict the optimal number of cells to infuse due to variations of T cell responses, persistence and side effects in different patients (11). Hence, there is a great need to engineer CAR T cells that can be controlled with high precision in space and time.

To this end, suicide switches and inhibitory corticosteroids have been applied to disable the infused CAR T cells in case of an adverse event (12, 13). However, these off-switches are designed to permanently down-regulate the activity of CAR T cells, which will limit the desired tumor killing. Alternatively, split-CARs have been engineered to assemble into fully functional CARs in the presence of a small molecule drug (3). Inhibitory CARs (iCARs), synNotch, as well as an elegant split, universal and programmable (SUPRA) CAR were also developed (14-16) to provide more control over the timing and dosage of CAR T therapy (13). However, these small molecule-based dimerization strategies lack confined tissue specificity, largely due to the diffusion of the small molecules. The temporal resolution is also limited by the time required for cell permeation and diffusion, which is partly attributed to the difficulty to deliver these molecules to specific tissue sites for in vivo studies (17). By contrast, genetically encoded dimerizers utilizing photoreceptors can allow localized, non-invasive and reversible control of protein-protein interactions by light with high spatiotemporal resolution, making them ideal candidates for gene regulatory systems (18-22).

We therefore aimed to develop controllable on-switch CAR T cells which can be activated by light stimulation at a confined tissue space, addressing the critical issue of “on target, off tumor” in CAR T immunotherapy. Red or infrared light systems have deep tissue penetration due to their long wavelengths, and would be ideal for therapeutic applications (22). However, most of them require exogenous supply of chromophore or expression of several bacterial or plant enzymes (20, 23, 24). Moreover, our initial tests with the PhyB-PIF6 red light system (20) did not achieve robust light-activatable gene expression, preventing us from utilizing these systems. We then switched to the blue light-based CRY2-CIB1 system, which has been reported to be rapid, reversible and highly sensitive to blue light stimulation, and is suitable for controlling gene expression (17-19, 25, 26). However, the CRY2-CIB1 pair has a high background in heterodimerization. Here we developed a light-inducible nuclear translocation and dimerization (LINTAD) gene activation system, integrating the CRY2-CIB1 pair in combination with the LOV2-based light-inducible nuclear localization signal biLINuS to reduce the intrinsic background noise from the non-specific CRY2 and CIB1 dimerization. We demonstrated that the LINTAD system with pulsed light stimulation patterns can regulate the expression of various target functional genes in multiple mammalian cell lines. Moreover, we improved the LINTAD system to achieve optimal gene regulations at low copy numbers of genetic cassettes, a condition compatible for cellular functions in primary human T cells. These T cells engineered with improved LINTAD system and inducible CAR achieved non-invasive control of tumor cell killing both in vitro and in vivo. The LINTAD system developed in this work can hence be used as a general tool for gene regulation, with applications in controllable cell-based cancer immunotherapy.

### Results

### Design of the Light-Inducible Translocation and Dimerization System for Gene Activation

The light-inducible dimerization of CRY2-CIB1 has enabled the development of gene activation systems that respond to blue light (18, 25). With a DNA-binding domain and a transcription activator fused to CRY2 and CIB1, respectively, the light-induced dimerization of CRY2-CIB1 can bring the transcription activator to the promoter to activate gene transcription and expression. However, there is a significant leakage of the transcriptional activities originating from the spontaneous binding between CRY2 and CIB1 even in the absence of light (18, 25). Konermann et al. fused nuclear localization signal (NLS) to CRY2 and nuclear export signal (NES) to CIB1 to spatially separate these two components before light stimulation to reduce the background, hence the residential CRY2-NLS would recruit NES-CIB1 to dimerize in the nucleus only in response to light (19). However, the forced retention of CIB1 outside of nucleus by NES could compromise the nuclear translocation efficiency and transcriptional activation power upon light stimulation.

Here, we develop a new light-inducible gene activation system LINTAD which combines CRY2-CIB1 dimerization and light-inducible nuclear translocation. We fused the LexA DNA-binding domain (17) to the N-terminus of CIB1 (19) and a bipartite light-inducible nuclear localization signal (biLINuS (17)) to its C-terminus, resulting in LexA-CIB1-biLINuS or LCB (FIG. 1a). Meanwhile, we fused an NLS to the N-terminus of

CRY2PHR (photolyase homology region of Arabidopsis CRY2, amino acids 1-498) (25) and a strong transcription activator VPR (27) to its C-terminus, resulting in CRY2-VPR or CV. Light-inducible reporters were constructed by assembling the LexA binding sequence (LexA BS), a minimal promoter, and a target reporter gene. In the dark state, LCB stays in the cytoplasm while CV stays in the nucleus, thus these two regulators at rest are spatially separated in different cellular compartments (FIG. 1a). Upon blue light stimulation, the biLINuS in LCB is activated, unfolding the Jα helix of LOV2 domain and exposing its NLS motif (17) to cause the nuclear translocation of LCB (FIG. 1b). Simultaneously, the CRY2PHR domain in CV is activated by blue light and can bind to the CIB1 domain of LCB with high affinity (18, 19). The LCB-CV complex is hence targeted to the LexA BS on the reporter cassette so that VPR is in close proximity to the minimal promoter, triggering transcription of the target reporter gene (FIG. 1b). This “push-pull” strategy incorporating the biLINuS-enabled active nuclear translocation of LCB as well as the pulling effect of the CRY2-CIB1 dimerization is expected to cause strong gene activation upon blue light stimulation with a high signal/noise ratio.

### Characterization of LINTAD Gene Activation System in HEK 293T Cells

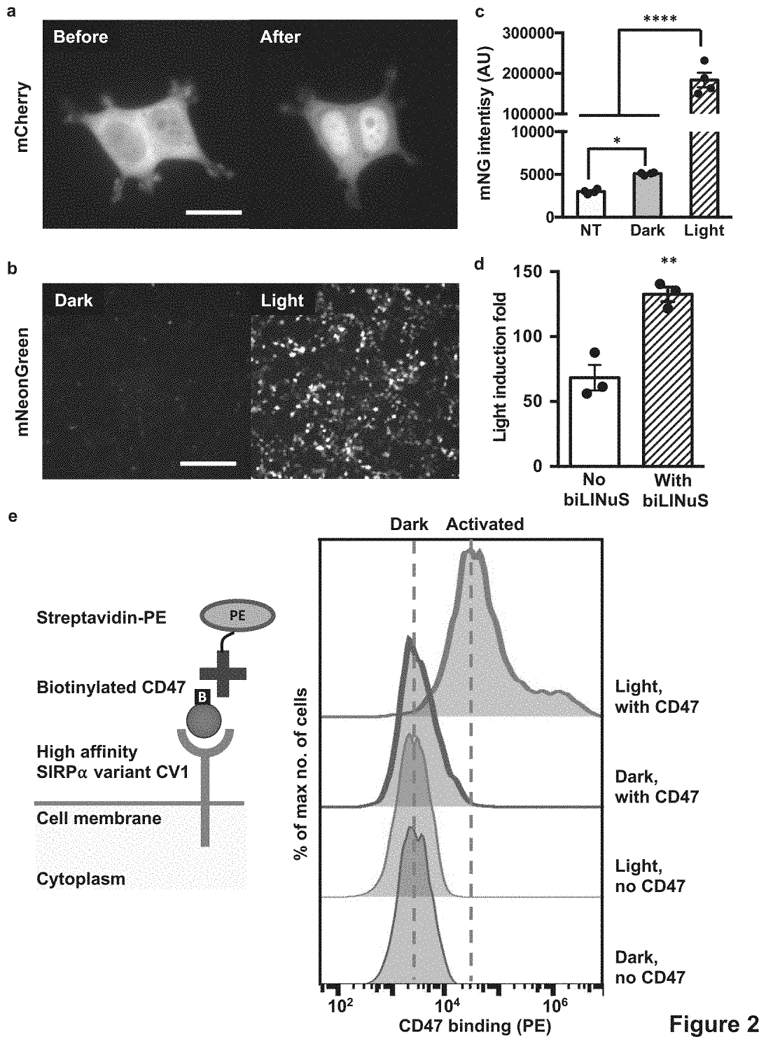

We first characterized the LINTAD gene activation system in HEK 293T cells. To visualize the blue light-induced translocation of LCB, the mCherry fluorescent protein was incorporated in LCB for tracking subcellular translocations (LexA-CIB 1-mCherry-biLINuS or LCmB). Indeed, the majority of LCmB showed cytosolic localization before blue light stimulation, and short pulses of blue light (repetitive 0.5 s light per 30 s for 10 min) triggered a clear nuclear translocation (FIG. 2a; Movie S1). The observed nuclear translocation was also verified to be specifically sensitive to blue light illumination (Fig. S1a-b), which will be referred to as “light stimulation” in the remainder of this manuscript if without further specification. The nuclear localization of CV was also verified (FIG. S1c). To examine whether LINTAD can activate gene expression upon light stimulation, HEK 293T cells were co-transfected with LCB and CV-expressing plasmids together with a light-inducible mNeonGreen reporter. Twenty-four hours after transfection, cells were either stimulated with pulsed blue light (5 mW/cm², 1 s per every 30 s) or kept in dark for 24 hr. Both fluorescence imaging and flow cytometry results showed that the light stimulation caused a significant induction of mNeonGreen, with the quantified mean cellular fluorescence intensities being 61-fold and 1.7-fold in the light and dark groups, respectively, compared to the non-transfected cells (FIG. 2b-c; Movie S2). These results demonstrate that the LINTAD system can control target gene expression through light stimulation in HEK 293T cells. To investigate the effect of including biLINuS in the LINTAD system, a similar light-inducible dimerization system without biLINuS (i.e., LexA-CIB1 and CRY2PHR-VPR) was constructed and compared with the LINTAD system using firefly luciferase (Fluc) as the reporter gene. Cells were also transfected to constitutively express Renilla luciferase (Rluc) to normalize the induced Fluc activity of each sample (dual-luciferase reporter system (28), see FIG. S2a). The LINTAD system showed a 133-fold induction of

Fluc activity normalized by Rluc upon light stimulation compared to the dark control, while the system without biLINuS showed a 68-fold induction (FIG. 2d). These results confirmed that biLINuS in the LINTAD system can significantly enhance the dynamic range of the blue-light-mediated gene induction. In addition, the LINTAD system had minimal background expression in the dark state which is comparable to the control systems where either the LCB or the CV component is removed (FIG. S2b), suggesting that the gene activation of LINTAD can be well controlled by light with high specificity.

We further explored whether LINTAD can be used to control gene expression for the regulation of cellular functions. CV1, an engineered high-affinity membrane receptor for CD47(29), was used here as the target gene in the light-inducible reporter, which can alter the cell's inherent CD47 binding behavior (29). The light-induced functional CV1 can be measured by staining with biotinylated CD47, which can then be detected by streptavidin-PE after extensive wash steps (scheme shown in FIG. 2e, left). More than 80% of the engineered cells upon light stimulation can be successfully stained with CD47 (FIG. 2e, right), in contrast to the minimal CD47 staining of the cells without light stimulation, which is similar to the controls without CD47 staining. These results indicate that LINTAD can successfully control membrane receptor production, such as CV1, for the regulation of cellular functions.

### Optical Control of Anti-CD19 CAR Expression in Jurkat T Cells

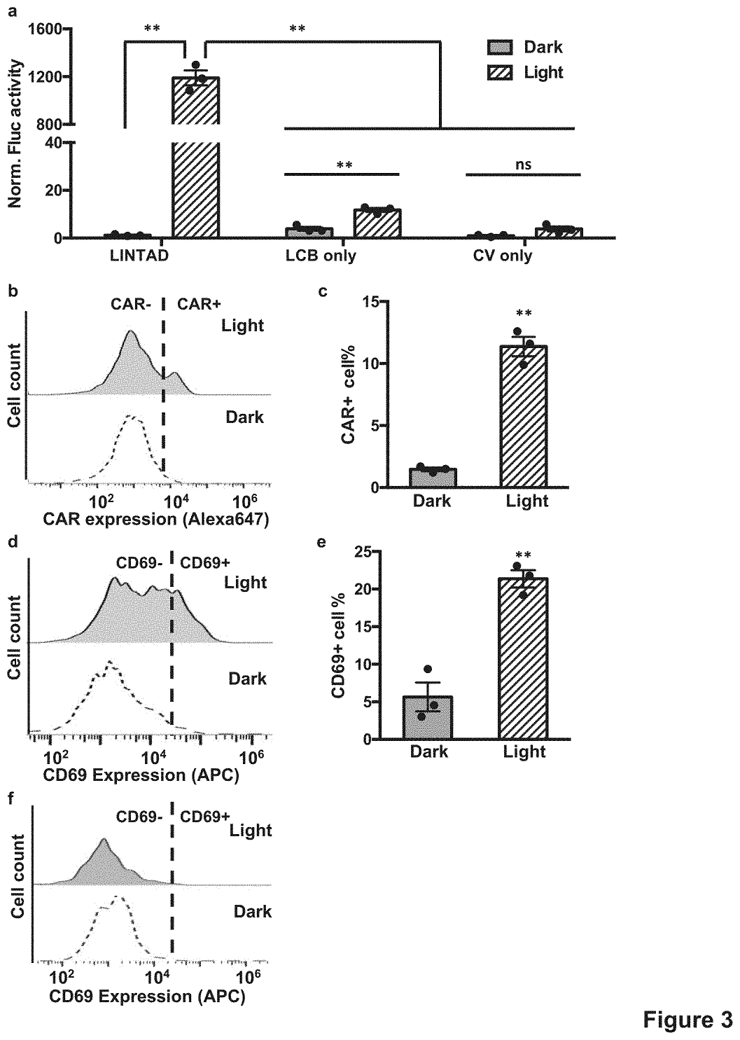

We further characterized LINTAD in human Jurkat T cell line, where T cell signaling can be activated by CARs (3, 16). To demonstrate that gene activation can be achieved upon light stimulation in Jurkat cells engineered with LINTAD, Fluc was used as the inducible reporter gene. We observed an over 1,000-fold increase of Fluc signal in the light-stimulated group comparing to the dark group (without light stimulation), with minimal background leakage expression (FIG. 3a). Next, we examined the capability of LINTAD in controlling anti-CD19 chimeric antigen receptor (CD19CAR) expression in Jurkat cells with the light-inducible Myc-tagged CD19CAR reporter. We observed significant induction of CD19CAR expression (quantified by the percentage of CAR positive cells) upon light stimulation (FIG. 3b-c). The detected induction fold appeared lower than that of the luciferase reporter, possibly due to the amplification effect of luciferase assays, the membrane expression characteristics and the increased gene size of CD19CAR (30) compared to Fluc. Other light-inducible reporters were also tested (mNeonGreen and CD19CAR-YPet) and showed similar light-mediated gene induction (FIG. S3). We noticed that when gene induction was quantified by fluorescent protein intensity, the noise level (cells with low fluorescent intensity in a sample) decreased slightly after light stimulation as compared to the sample without light stimulation (FIG. S3c-d), possibly due to the photo-bleaching effect of light stimulation (FIG. S1d-e). When the light stimulation is stopped, the CRY2-CIB1 interaction in the LINTAD system would dissociate with a half-life of approximately 5.5 min (18, 31), and hence there is no further transcription/translation of the reporter gene. Therefore, the duration of the induced gene expression mainly depends on the degradation of the proteins encoded in the reporter genes. In our study, the duration of the light-induced CAR expression is approximately 2 days (FIG. S3e).

Functionality of the light-induced CD19CAR on these Jurkat cells was verified by co-culturing with Toledo cells (a human non-Hodgkin's lymphoma cell line), which express CD19 antigen and are expected to trigger the CD19CAR-mediated T cell activation signaling in Jurkat cells. Indeed, we observed more than three-fold elevated CD69 expression (an early T cell activation marker (32)) in the light-stimulated group comparing to that of the control group without light stimulation (FIG. 3d-e), suggesting that the light-induced CD19CAR can enable Jurkat cells to recognize CD19 antigen and activate the corresponding signaling pathway. To further verify that this activation is CD19-recognition dependent, we constructed a reporter containing a truncated version of CD19CAR, where the CD19-recognizing extracellular domain was deleted (headless CAR). The expression of this headless CAR can also be induced by light (FIG. S3f), but was not able to induce the upregulation of CD69 expression after co-culturing with Toledo cells (FIG. 3f), indicating that the observed light-induced cell activation is specifically mediated by CD19 recognition. Our results hence demonstrated that LINTAD can non-invasively induce gene expression in Jurkat cells with light, including not only the fluorescent protein reporters but also the functional CAR molecules, revealing the potential of optogenetics in cell-based immunotherapy.

### Light-Inducible Cytotoxicity of Engineered Primary Human T Cells

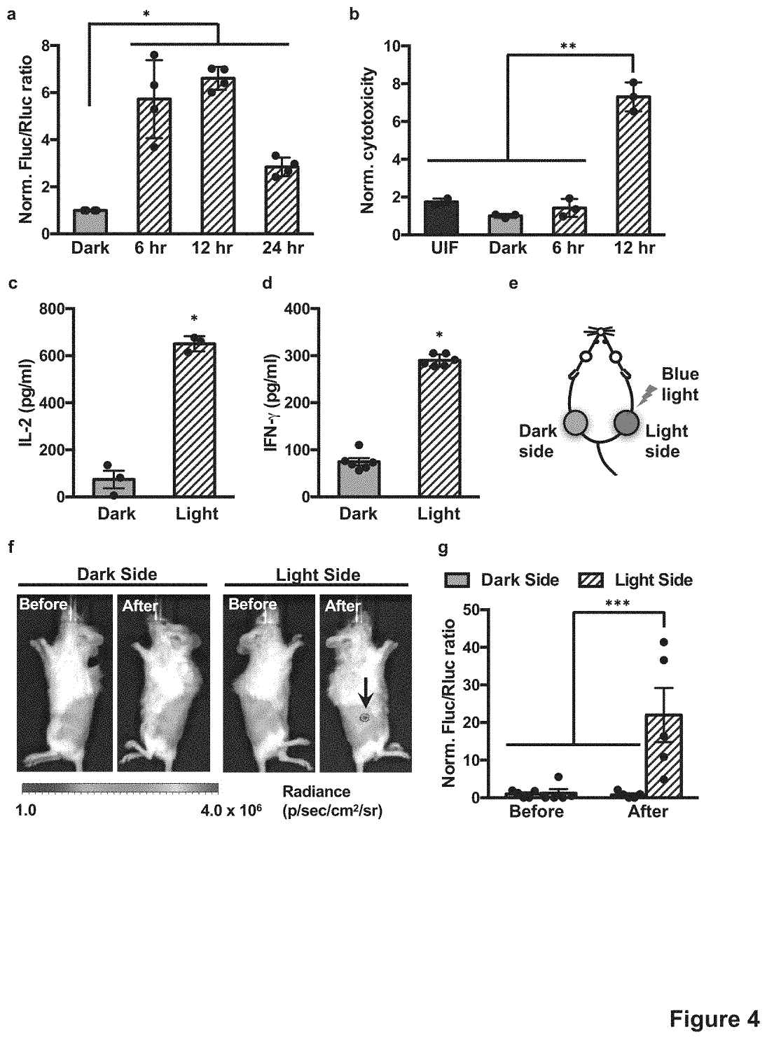

To demonstrate the light-controllable cell-based immunotherapy, we tested our system in primary human T cells by lentiviral transduction of the LINTAD regulators together with corresponding reporters (FIG. S4a-c). We first used the dual-luciferase reporter containing a light-inducible Fluc and a constitutive Rluc (as internal reference), and observed 2-7 folds of gene induction with different durations of light stimulation (1 s light per 30 s, for 6-24 hr, FIG. 4a), with 12 hr light stimulation resulting in the highest induction, demonstrating that blue light can be used to control gene expression in primary human T cells. We then focused on light-inducible CD19CAR expression and tested whether we can achieve light-inducible cytotoxicity of T cells against target tumor cells. T cells infected with LINTAD regulators and CD19CAR reporter (FIG. S4d, with regulators and reporter fused to fluorescent proteins to assess the gene transduction efficiency in FIG. S4e) were stimulated by light for 6 or 12 hr, followed by co-culture with target tumor cells (Nalm-6 cells expressing CD19 and engineered to constitutively express Fluc) for 24 hr. Cytotoxicity of T cells were determined by measuring the Fluc activity of the remaining live Nalm-6 cells after co-culture in each group. The engineered T cells, only after 12 hr light stimulation, demonstrated 7.3-fold enhanced cytotoxicity compared to that of the control group which was kept in dark or that of the uninfected T cells (FIG. 4b), suggesting that there is a threshold of stimulation time to induce adequate expression of CD19CAR for enhanced functional actions. To verify that the cytotoxicity is specific to T cell activation, the levels of the cytokines interleukin 2 (IL-2) and interferon y (IFN-γ) in the supernatants after co-culture were quantified (15). Higher secretion of both cytokines (8.8-fold and 3.9-fold, respectively) was observed in the 12 hr light stimulation group compared to the dark group (FIG. 4c-d). Our results hence suggest that light-inducible cytotoxicity of CAR T cells can be achieved through LINTAD.

### LINTAD-Mediated Gene Induction In Vivo

The skin/muscle penetration of blue light has been a concern for its in vivo therapeutic use. To explore whether our LINTAD system can be applied in vivo, Nalm-6 cells engineered, by lentiviral infection, with LINTAD and the dual-luciferase reporter (see Materials and Methods and FIG. S4c) were subcutaneously injected on both sides of shaved mice (FIG. 4e), of which only one side was subjected to short-pulsed blue light stimulation using an LED patch controlled by a home-built light box (light side, 1 s light per 30 s, see FIG. S5 and Materials and Methods), while the other side remained unstimulated (dark side). After 24 hr of light/dark treatment, the light side showed significant gene induction (Fluc/Rluc), while the dark side had minimal Fluc signal (FIG. 4f-g). This result demonstrates that blue light can penetrate skin tissue in vivo to achieve gene induction via LINTAD.

### In Vivo Cytotoxicity Of Light-Stimulated Primary Human T Cells Engineered With LINTAD

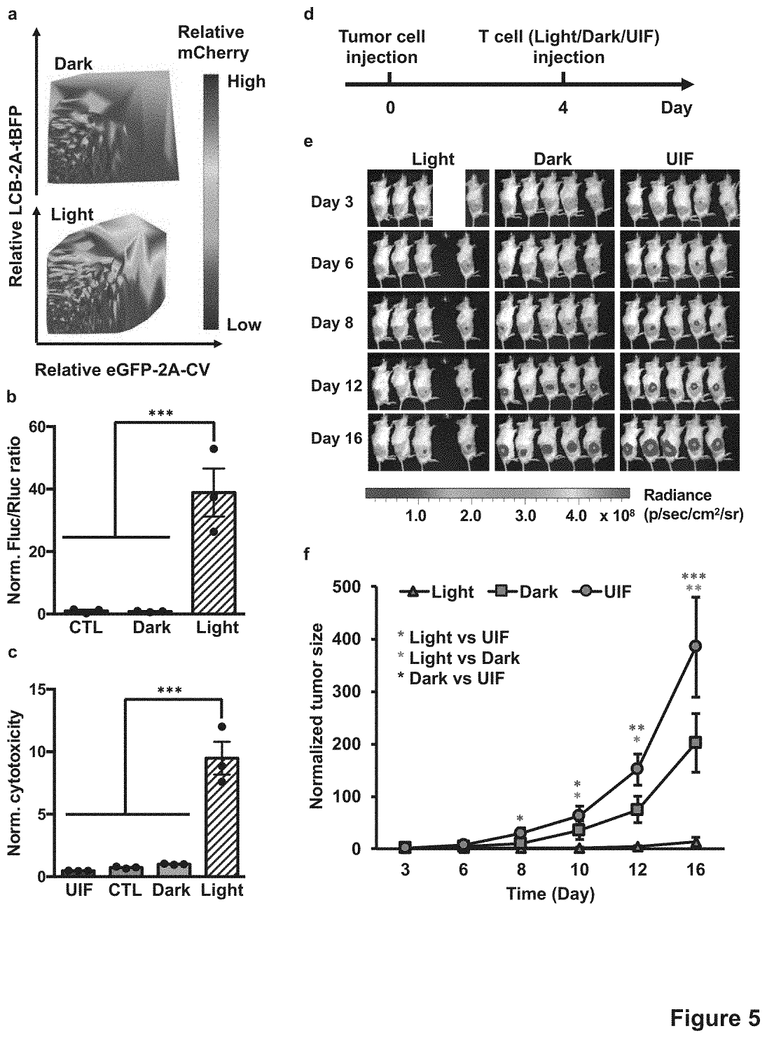

As we were working to characterize and optimize LINTAD for improved induction efficiency in T cells, we found that there is a strong correlation between the cellular concentrations of LINTAD regulators (LCB and CV quantified by the fused tBFP and eGFP intensities, respectively) and the resultant light induction efficiency (quantified by the reporter mCherry expression) (FIG. 5a and FIG. S6a-c). Only when the concentrations of regulators (LCB or CV) reach a threshold can the reporter gene be efficiently activated, with the induction efficiency peaked at the highest concentration zone for both regulators. In contrast to HEK 293T or Jurkat cell line, the relatively low expression levels of regulators LCB and CV based on lentiviral infection in primary human T cells severely limited the gene induction efficiency. The required high concentration of regulators for efficient gene induction may suggest that the affinity between CRY2 in CV and CIB1 in LCB is relatively low even after light activation. One way to enhance their binding is to engineer a pair of dimerization helpers into the current system, attaching to the LINTAD regulators LCB and CV, respectively. To this end, several helper pairs with diverse affinities were tested using different fusion strategies (33, 34) (FIG. S7a). Among the different designs tested, the trend of WW-WP1 and SH3-SP1 helper pairs (35) were identified to favor a higher light induction efficiency when the regulators are at low concentrations (FIG. S7b). This was further verified by the light-induced luciferase assay in primary human T cells (FIG. 5b and FIG. S8). Specifically, the WW-WP1 pair caused a significant improvement over the original LINTAD system in light-induced gene activation of Fluc (approximately 40 fold in FIG. 5b vs. approximately 7-fold in FIG. 4a), which was hence used for further experiments in primary human T cells.

With this improvement, we applied LINTAD with the WW-WP1 addition (WW-LINTAD) for light-inducible cell-based immunotherapy in vivo. In order to maintain a more sustained CD19CAR expression after light is off, a Cre-LoxP system was utilized here, where the light-induced Cre can turn on CD19CAR expression in the loxP-ZsGreen-stop-loxP-CD19CAR cassette (FIG. S9), so that a short-pulsed transient light stimulation (hours) can be converted into a long-lasting CD19CAR action (days to weeks). The engineered T cells with the WW-LINTAD and Cre-LoxP system upon 12 hr light activation indeed demonstrated a significant 9.5-fold light-inducible cytotoxicity in vitro (FIG. 5c). To test their light-induced cytotoxicity in vivo, these engineered T cells after in vitro light activation were subcutaneously injected into NSG mice 4 days after subcutaneous tumor inoculation (FIG. 5d). Bioluminescence imaging (BLI) was performed to monitor tumor burden for 16 days. The tumor growth in the mice injected with the light-stimulated engineered T started to show significant difference from the control group (injected with uninfected T cells) on Day 8. The significant difference between the light and dark (engineered T cells without light stimulation) groups started to reveal on Day 12 (FIG. 5e-f), demonstrating the cytotoxicity effect of light-induced T cells against tumor cells in vivo.

### In Vivo Light-Inducible Immunotherapy with High Spatial Resolution

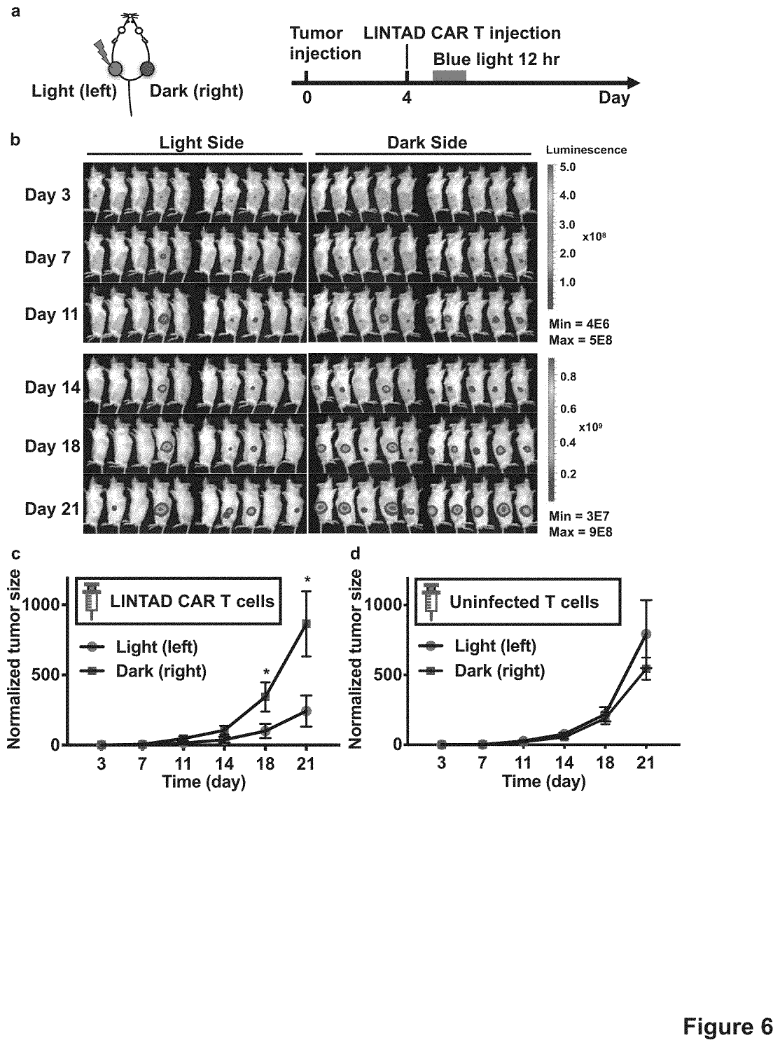

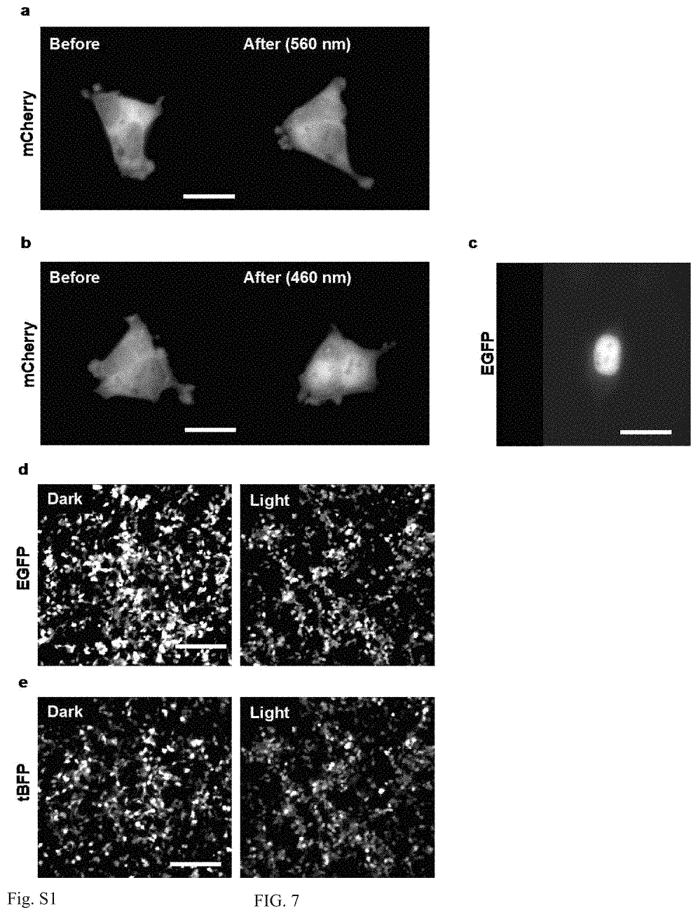

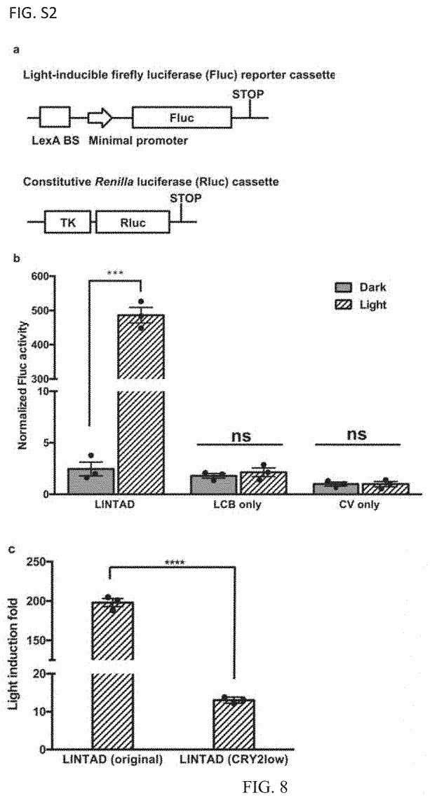

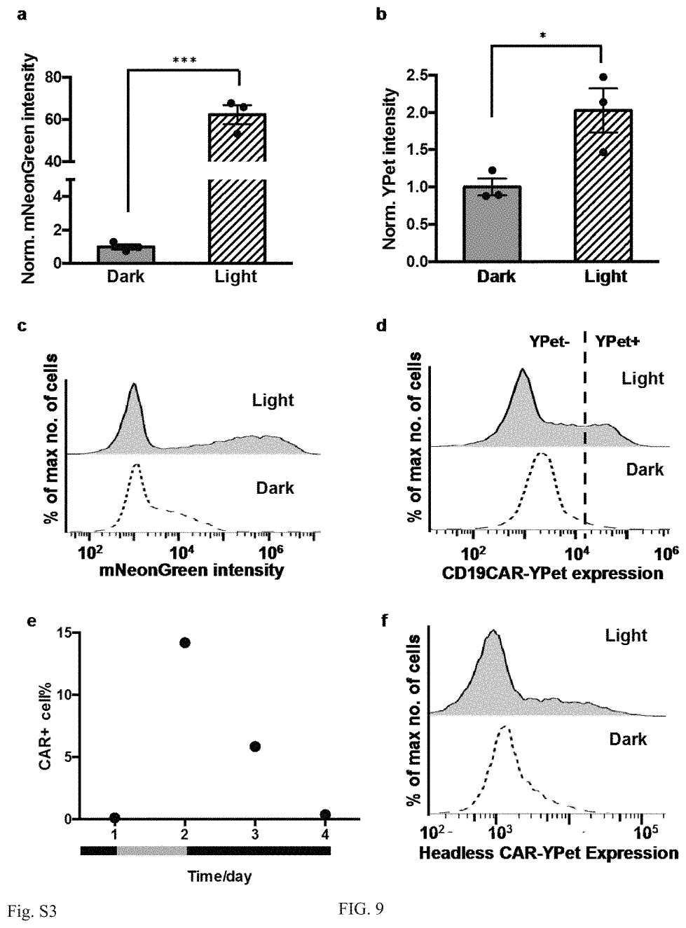

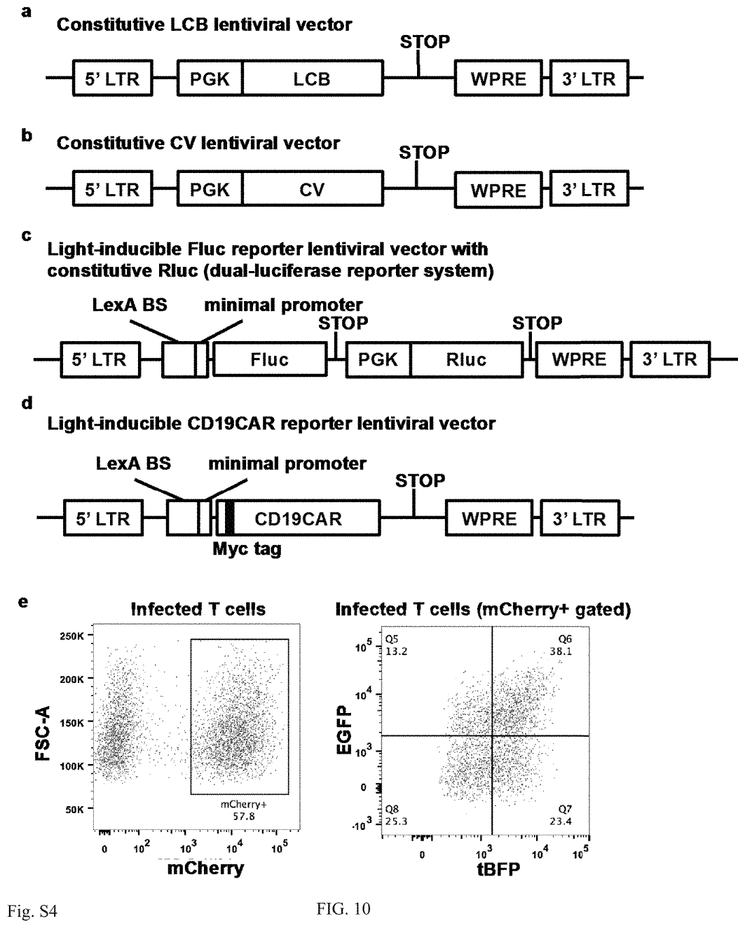

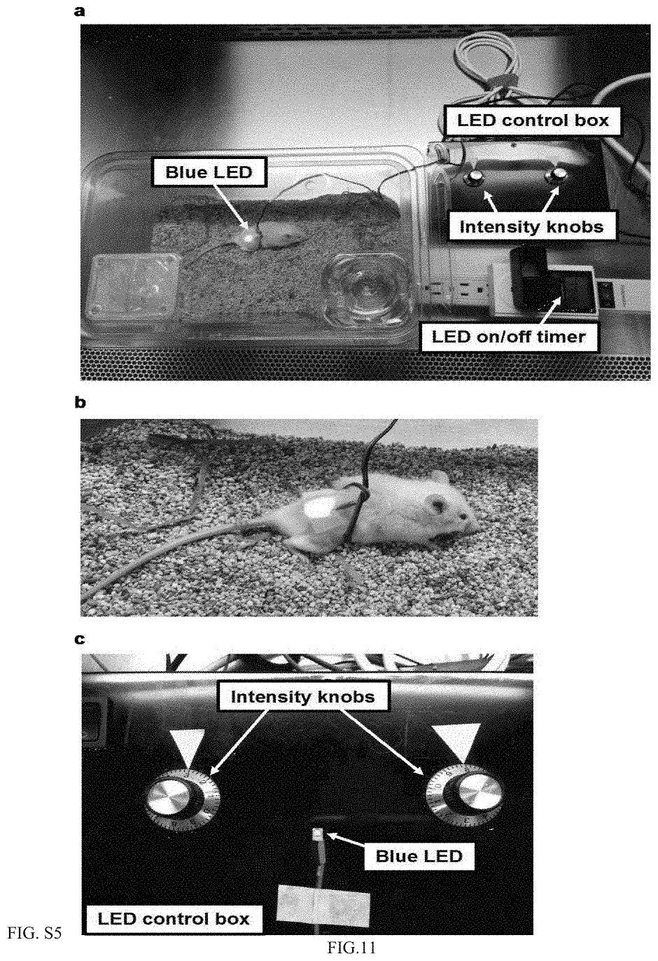

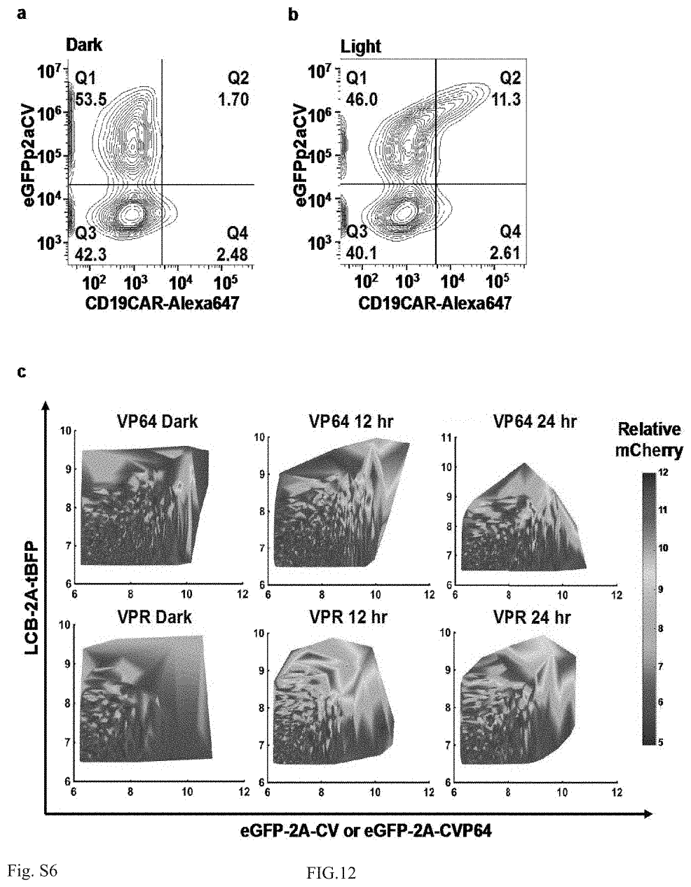

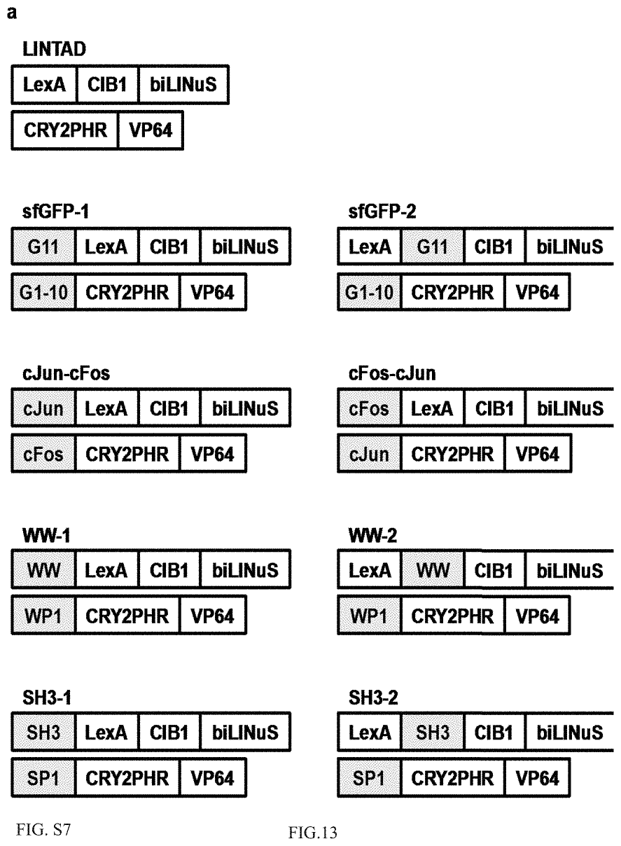

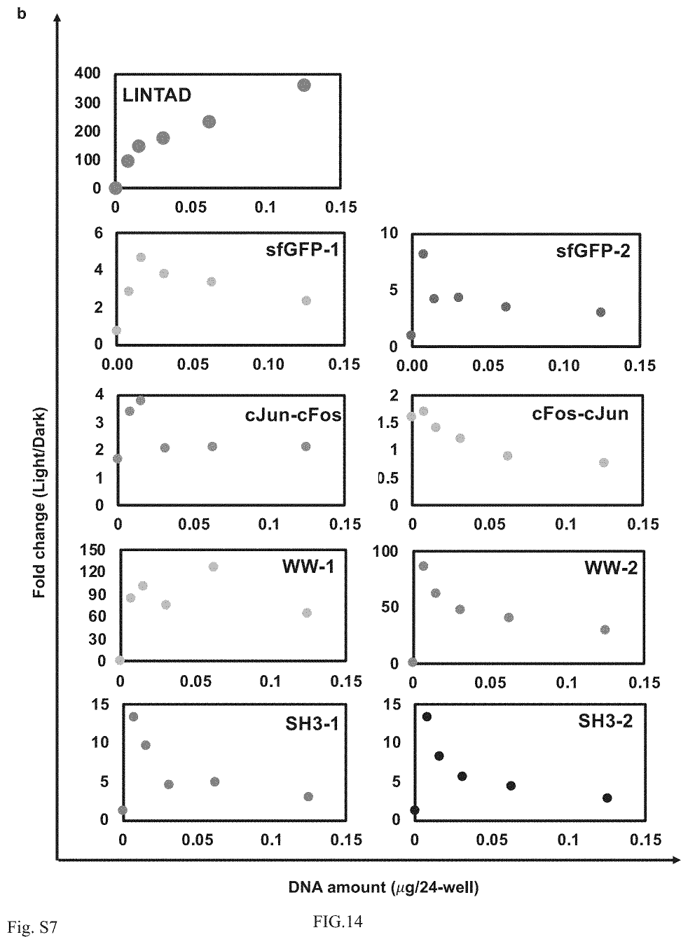

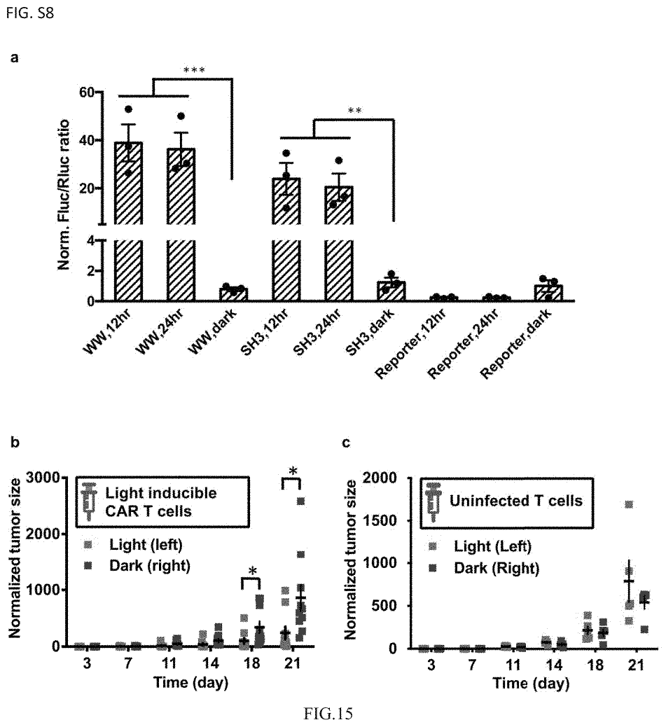

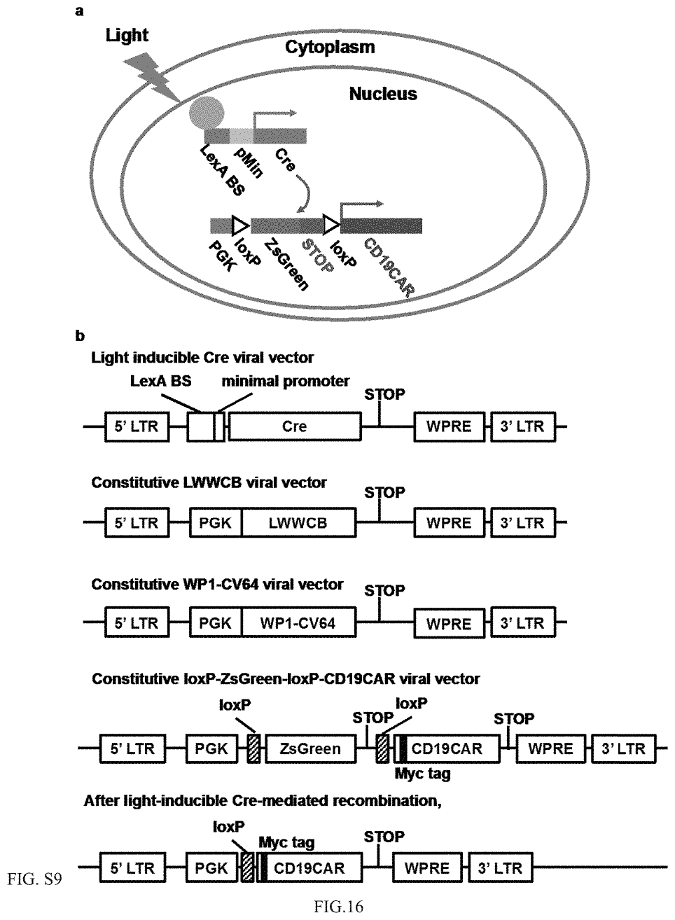

We reasoned that our engineered light-inducible CAR T cells after injection can be activated in vivo with high spatial resolution, providing controllability of CAR T immunotherapy as compared to the standard CAR T therapy. To demonstrate this, NSG mice were subcutaneously inoculated with Nalm-6 tumor cells (expressing Fluc) on both left and right flanks. Four days later, engineered T cells with WW-LINTAD and Cre-LoxP-based inducible CD19CAR were locally injected into the mice at both tumor sites (FIG. 6a). The tumor on one flank received blue light stimulation in vivo (Light), while the tumor on the other flank remained un-stimulated (Dark) as described in Materials and Methods. Tumor aggressiveness was monitored for three weeks by BLI. We observed that the tumor growth on the light side was significantly inhibited compared to that on the dark side (FIG. 6b-c, FIG. S8b), demonstrating the desired spatial selectivity and controllability of the light-inducible cytotoxicity in vivo. To demonstrate that the light-induced tumor inhibition was through LINTAD-controlled CD19CAR, we injected non-engineered primary human T cells locally into the tumors on both flanks in NSG mice, and applied the above-mentioned light or dark treatment. No significant difference in tumor growth was found between the light and dark sides, suggesting that blue light itself does not affect tumor aggressiveness (FIG. 6d, FIG. S8c). Taken together, our results show that the LINTAD-based light-inducible CAR T cells can be activated in vivo by blue light at the desired location with spatial selectivity to achieve therapeutic effects, which provides spatiotemporal control of cell-based immunotherapy.

### Discussion

Cell-based immunotherapies such as CAR T cells are becoming a paradigm-shifting therapeutic approach with promising success in treating blood tumors. With the next frontier of applying cell-based therapies in solid tumors, one critical component of technology development is to gain spatiotemporal control of engineered T cells with high precision (3, 13, 16). The LINTAD system developed here demonstrates the first successful integration of optogenetic control and CAR T, and provides the proof of concept for light-controllable immunotherapy, which has the potential to precisely address this need particularly for tumors at shallow surfaces including skin cancers and melanoma. Although red or infra-red light systems have the advantage of deeper tissue penetration, most of them at the moment require additional co-factors besides photoactivatable domains and appear less efficient than the counter parts of the blue light systems (23, 36). The limited tissue penetration of blue light (37) can also be overcome to reach deeper tissues using upconversion nanoparticles which are capable of converting near-infra-red (NIR) light to stimulate blue-light-responsive proteins (38), or implantable light emitting diodes controlled wirelessly with radio frequencies (39) or MR light (40). Integrated with optical fibers, LINTAD can also be extended to control engineered T cells targeted at gastrointestinal tracts to treat gastrointestinal cancers (41). As this is a rapidly evolving field, LINTAD could provide the power of controlling engineered cells with high precision for cell-based therapeutics to eradicate tumors, particularly for these locally advanced and unresectable solid tumors.

While the CRY2-CIB1 dimerization has been utilized to engineer various gene activation systems, the basal dimerization tendency of the CRY2-CIB1 pair can result in a relatively high level of leaky expression of reporter genes (18, 25). Efforts on directed evolution attempted to suppress the basal expression and improve the dynamics for gene regulation have met with limited success (25). Another strategy to minimize the basal leakage is to separate CRY2 and CIB1 into different cellular compartments before light stimulation (19). However, the nuclear translocation relies on the equilibrium between the strengths of the localization signals (NES and NLS) on these components when they form a complex, thus resulting in limited shuttling of the molecules between the compartments and therefore limited activation efficiency. Our LINTAD system reported here utilizes both the light-induced active transportation via the biLINuS motif and the pulling effect mediated by the CRY2-CIB1 dimerization for nuclear translocation to control gene activation. Without light, CIB1 stayed outside of the nucleus and was decoupled from CRY2 to achieve a low basal leakage of target gene expression. Upon light activation, this “push-pull” strategy combining the biLINuS-enabled nuclear translocation and the CIB1-CRY2 dimerization induced a strong gene activation. As such, the LINTAD system can achieve a high contrast of gene activation before and after light stimulation.

The positive correlation between efficiency of light induction and expression levels of regulators of LINTAD system (FIG. 5a and FIG. S8) and the requirement of high expression levels of LINTAD regulators for efficient induction of reporter genes suggest that the binding affinity between CRY2-CIB1 may be weak even after activated by light. Engineering a weak dimer helper pair into the original LINTAD system indeed led to significant enhancement of the performance of LINTAD system, especially when the regulator concentrations are limited at low levels in primary human T cells (FIG. 5b). This improvement further led to in vivo cytotoxicity and tumor growth suppression upon light stimulation (FIG. 5e-f and FIG. 6). These results suggest that we can tailor the inducible dimerization partners in their affinity to optimize the system performance in different cell types with their special requirement of regulator concentrations. This approach can be particularly useful for the genetic engineering of primary cells where the exogenous protein expression levels are restricted.

The modular design of the LINTAD system allows a simple change of the DNA binding domain, the dimerization pair, and/or the transcription activator to integrate different molecular interactions for the control of desired protein production and functional cellular outcomes. Replacing the reporter gene with different target genes can conveniently switch, in principle, any functional outcome of LINTAD to monitor or reprogram host cells. For example, TRAIL (TNF-related apoptosis inducing ligand), an inducer of apoptosis and a cancer therapeutic (42), can be used as the target reporter gene and LINTAD can be utilized to achieve light-inducible tumor suppression/killing.

In alternative embodiments, the reporter genes also are regulators of CRISPR (clustered regularly interspaced short palindromic repeats), such as Cas9 and dCas9. Light and LINTAD can hence be applied to control genome editing with high precision in space and time.

The DNA binding domain LexA can be replaced by other types of its kind to orthogonally target different promoter sequences and drive gene products (18, 25), whose expression levels can also be fine-tuned through promoter sequence engineering (43). The VPR transcription activator can also be switched to smaller domains such as VP64 to reduce the gene cargo size for enhanced delivery efficiency in primary cells which are usually difficult to transfect or infect (19, 27). As such, we have engineered and characterized a new light-controllable modular system for gene activation, with their immense therapeutic potential demonstrated by the proof-of-concept application in engineered T cells for cancer immunotherapy. In our demonstration of the concept of light-inducible immunotherapy, the Cre-LoxP system was employed to convert transient light stimulation to relatively long-term CAR expression once the engineered T cells were activated. For situations where higher temporal resolution is desired, the reversibility of the current system can be tuned by incorporating other synthetic biology strategies (13) such as the degron motifs (44), which are known to regulate protein degradation rates and hence can control CAR T cells at the protein level. Alternatively, as the half-life of CAR molecules has been reported to be approximately 8 hours (hr) (16), the LINTAD system without Cre-LoxP can be directly used to trigger transient CAR expression, and multiple rounds of light stimulation can be applied to produce pulsed expression profiles of CAR with tunable magnitudes for more precise controls and mitigating CAR T cell exhaustion.

While similar therapeutic results achieved here using the locally injected and illuminated light-inducible CAR T cells could potentially be obtained with locally injected constitutive CAR T cells, the development of light-controllable CAR T cells can lead to a platform technology and be integrated with intravenous injection in the future to activate engineered T cells only at tumor sites by local illumination. LINTAD can be further integrated with bispecific antibody or a homing receptor on the cell surface so that intravenously introduced engineered T cells can accumulate at tumor sites. In fact, several groups have successfully used engineered T cells to identify and highlight tumor positions (45, 46).

CRY2 has been shown to simultaneously undergo CRY2-CRY2 homo-oligomerization in the CRY2-CIB1 dimerization system upon light stimulation (47). To minimize the potentially undesired CRY2-CRY2 homo-oligomerization which may affect the gene activation efficiency of LINTAD, we have engineered a version of LINTAD where CRY2PHR was replaced by the recently reported CRY2low (47), a CRY2 mutant with reduced homo-oligomerization. However, this did not result in any improvement when compared to the original LINTAD system which uses CRY2PHR (FIG. S2c).

### Materials and Methods

Cloning. Plasmids used in this manuscript are listed in Table S1. LexA-CIB1-biLINuS constructs were generated by Gibson assembly (New England Biolabs) of fragments LexA (from pDN92 (17)), CIB1 (from LITE2.0 TALE-CIB1 (19)) and biLINuS (from pDN92 (17)) following the manufacturer's instructions. CRY2PHR-VPR constructs were generated by Gibson assembly of fragments CRY2PHR (from LITE2.0 CRY2PHR-VP64 (19, 48)) and VPR (from SP-dCas9-VPR (27)). DNA fragments were amplified by PCR using Q5 DNA polymerase (New England biolabs). DNA oligos for PCR amplification were synthesized by Integrated DNA technologies. PCR products were purified by agarose gel electrophoresis method (Zymo Research, D4001). Purified DNA fragments were cloned into pCAGGS vector (for transient transfection) or pHR-PGK (for transient transfection or lentiviral infection). Light-inducible reporter constructs were generated by cloning the target gene into the pDN100 vector (with original reporter gene removed, for transient transfection) or pHR vector (with LexA binding site and minimal promoter, for lentiviral infection) using Gibson assembly method. Constructs were confirmed by

### Sanger Sequencing (Genewiz).

Reagents and cell culture. Fetal bovine serum (FBS), DMEM, RPMI 1640, L-glutamine, penicillin/streptomycin and sodium pyruvate were purchased from Gibco. Phosphate buffered saline (PBS) was prepared from PBS powder (Sigma-Aldrich). Biotinylated CD47 protein was produced and purified as previously described (49). Cell lines HEK 293T (human embryonic kidney 293T cell line), Jurkat (human acute T cell leukemia cell line) and Toledo (human non-Hodgkin's B cell lymphoma cell line) were from American Tissue Culture Collection (ATCC, Manassas, Va.), with the authentication and verification of the absence of mycoplasma contamination. Nalm-6 cell line (engineered to express firefly luciferase) was a gift from Michel Sadelain Lab. These cells were cultured in ATCC-recommended conditions in a humidified incubator of 95% air and 5% CO₂ at 37° C.

Live cell imaging of light activated protein nuclear translocation. 0.8 million HEK 293T cells were seeded in a 3.5-mm dish, transfected at 70% confluency with 2.0 μg of pcDNA3.1-LCmB, and cultured in dark (covered by aluminum foil; same for other experiments unless otherwise specified). Twenty-four hours after transfection, medium was replaced with phenol red free DMEM with 10% FBS for imaging. Live cell imaging was performed at 37° C. with a Nikon Ti inverted microscope equipped with a ×100/NA 1.4 objective and a cooled charge-coupled device camera (Cascade 512 B,Photometrics) using MetaFluor 6.2 software (Universal Imaging). The dynamic nuclear translocation of mCherry in LCmB was monitored by an excitation filter 580/20 nm and an emission filter 630/20 nm, together with a dichroic mirror 595 nm (Chroma). Blue light stimulation was delivered with an excitation filter 465/30 nm.

In vitro blue light stimulation on cells. Unless otherwise specified, light simulation in vitro on all types of cells was achieved using a customized 460 nm blue LED lamp mounted to the inside of an above-mentioned standard cell culture incubator. The distance between the LED and the cell culture plate was measured and fixed so that the cells were exposed to a light intensity of 5 mW/cm². A programmable plug-in digital timer switch (Nearpow) was used to control the light stimulation pattern, for example, 1 s/30 s for 24 hr. The cells in the Dark groups were kept in cell culture plates covered by aluminum foil in a separate standard cell culture incubator.

Imaging of light-inducible gene expression of mNeonGreen in HEK 293T cells. Cells were transfected with LCB and CV-expressing plasmids and light-inducible mNeonGreen reporter plasmid, and placed in dark immediately after transfection. For light group, 24 hr after transfection, the cells were stimulated with the customized blue LED lamp (5 mW/cm², 1 s/30 s) as described above for 24 hr. For dark group, the cells were kept in dark until imaging. Images of mNeonGreen were taken at 48 h after transfection for the light and the dark groups with an excitation filter 460/40 nm, an emission filter 525/50 nm, and a dichroic mirror 495 nm (Chroma). Luciferase reporter assays in HEK 293T cells. 0.8 million cells were seeded in a 35-mm cell culture dish, at 70% confluency, cells were co-transfected with 1.5 μg of LCB, 1.5 μg of CV, 1.0 μg of light-inducible firefly luciferase (Flue) reporter, and 20 ng of a constitutive Renilla luciferase (Rluc) construct as a normalization reference (phRL-TK, Promega) using Lipofectamine 2000 (Invitrogen). Twenty-four hours after transfection, the cells were stimulated by blue light for 24 hr (5 mW/cm², 1 s/30 s) or kept in the dark. All the cells in each dish were harvested, washed, and lysed in passive lysis buffer (Dual-Glo luciferase assay kit, Promega), and Fluc and Rluc activities were quantified following manufacturer's protocol using a Tecan Infinite M200 Pro plate reader. CV1-CD47 binding assays in HEK 293T cells. Cells were transfected with LCB and CV-expressing plasmids and light-inducible CV1 reporter, and cultured in dark. For light stimulation, 24 hr after transfection, the cells were stimulated with blue light (5 mW/cm², 1 s/30 s) for 24 hr. The cells were trypsinized and incubated with or without biotinylated CD47 (49) in PBS for 30 min at room temperature on a rotator. Cells were then washed three times with PBS, and stained with streptavidin-PE in PBS for 30 min at room temperature on a rotator. After further washing three times with PBS, the cells were resuspended in PBS and analyzed with BD Accuri C6 flow cytometer for PE staining. Electroporation and light-inducible gene activation in Jurkat cells. Ten million Jurkat cells (in growth phase after 1:3 passaging the night before) were washed with OptiMEM (Gibco), and resuspended in 500 μL OptiMEM with 15 μg of pHR-PGK-LCB, 15 μg of pHR-PGK-CV, and 15 μg of various light-inducible reporters. The cell resuspension was transferred to a 4-mm electroporation cuvette, electroporated using the Bio-Rad Gene Pulser Xcell Electroporation System, at 270 V, 950 ↑F (exponential wave, infinite resistance), and immediately transferred to pre-warmed complete growth medium after electroporation. Twenty-four hours after electroporation, the cells were stimulated with blue light (5 mW/cm², 1 s/30 s) or kept in dark for 24 hr, and then analyzed for fluorescent protein expression using a flow cytometer (Accuri C6, BD). For luciferase assays, the reporter construct containing light-inducible firefly luciferase was used. The same number of cells after light or dark treatment were lysed followed by firefly luciferase measurement to quantify the light-induced gene expression. To measure the induction of Myc-tagged CD19CAR, the cells were stained using an Alexa Fluor® 647 conjugated Myc-tag mouse antibody (2233S, Cell Signaling Technology) following manufacturer's protocol and analyzed by flow cytometry. For CD69 expression assay, the cells were co-cultured with Toledo cells (ATCC CRL-2631) at 1:1 ratio for 24 hr, followed by anti-CD69 antibody staining (APC anti-human CD69, 310910, BioLegend) and flow cytometry analysis. For CD69 analysis, the YPet+populations of the co-cultured cells were used to exclude Toledo cells and Jurkat cells not containing all the three plasmids.

Isolation, culture, and lentiviral transduction of primary human T cells. Human peripheral blood mononuclear cells (PBMCs) were isolated from buffy coats (San Diego Blood Bank) using Ficoll gradients (Amersham Biosciences). CD3+ T cells were isolated from PBMCs using Pan T cell isolation kit (Miltenyi). For lentiviral transduction, cells were first activated for 72 hr using CD3/CD28-coated Dynabeads (Gibco) in complete RPMI medium with 100 IU/ml interleukin-2. The cells were then transduced with concentrated lentivirus cocktail at an MOI of 10 for each virus by spinoculation on Retronectin (Takara)-coated plates at 1,800 g, 32° C. for 1 hr. Light-inducible gene activation in primary human T cells. Primary human T cells were infected with lentiviral cocktail of the LINTAD regulators (LCB and CV) and the desired light-inducible reporter (Fluc/Rluc dual luciferase reporter or CAR) as described above. Infected T cells were kept in dark for 3 days before experiment. For light activation, T cells were illuminated using the above-mentioned blue LED for different durations of time (1 s/30 s, for 6 hr, 12 hr, or 24 hr). The inductions of different reporter genes were measured as described above.

Cytotoxicity assay of primary human T cells. The cytotoxicity of T cells was determined by luciferase-based assays (48). In brief, a Nalm-6 cell line expressing constitutive Fluc (a gift from Prof. Michel Sadelain) was used as the target tumor cells. Different groups of primary human T cells and Nalm-6 cells were co-cultured at 10:1 ratio in 96-well plates with 2×10⁴ target cells in a total volume of 200 μl complete RPMI medium per well.

Twenty-four hours later, the cells were centrifuged to remove the supernatant and lysed with 200 μL passive lysis buffer (Promega) according to manufacturer's instructions. Luminescence was then measured using a TECAN Infinite M200 pro plate reader. ELISA of IL-2 and IFN-γ. The BD ELISA kits for human IL-2 and IFN-γ(cat. #555190 and #555142) were used to quantify cytokine release in the supernatants after cytotoxicity assays following manufacturer's protocols.

In vivo blue light stimulation on mice. We have built 3 identical light boxes (FIG. S5), each containing two 460 nm blue LEDs with tunable intensities, to allow simultaneous light stimulation on multiple mice (maximum 6 mice at once). Six-to-eight weeks old male NOD.Cg-Prkdcscid Il2rgtm1Wjl/SzJ (NSG) mice were obtained from Jackson Laboratories and used for all animal studies described below under protocols approved by the UCSD Institutional Animal Care and Use Committee, and in accordance with ethical regulations and institutional guidelines. The mice were shaved prior to light stimulation, and the LEDs (half-sphere shaped with a diameter of approximately 2 mm) were attached to their skin at the stimulation sites using medical tapes (Nexcare). Light stimulation patterns were controlled digitally as described above.

In vivo Bioluminescence imaging. Bioluminescence imaging (BLI) was performed using an In vivo Imaging System (IVIS) Lumina LT Series III (PerkinElmer) and the images were analyzed using Living Image software (PerkinElmer). The luciferase activities of Fluc and Rluc were measured by their respective substrates D-Luciferin (GoldBio) and Viviren (Promega) following manufacturers' protocols. BLI of Fluc and Rluc of the same mouse was typically performed 4 hr apart to prevent interference between the two signals. Imaging settings were kept the same throughout the entire time course of an experiment. In vivo reporter gene activation by blue light. Wild type Nalm-6 cells were engineered through lentiviral infection to express LCB, CV, and the dual-luciferase reporter (inducible firefly luciferase and constitutive Renilla luciferase, LexA BS-Pₘᵢₙᵢ-Fluc-PGK-Rluc-P2A-mCherry). The engineered Nalm-6 cells were injected subcutaneously into both flanks of the mice (1×10⁶ cells/injection). The injected cells on one flank was stimulated with blue light (1s/30s, 24 hr) using the light box described above, while the skin around the injection site on the other flank was covered by a piece of aluminum foil as the dark control. The cages were covered by aluminum foil the entire time of the experiment. BLI of Fluc and Rluc were performed as described above, and the ratio of the integrated luminescence of the inducible Fluc to that of the constitutive Rluc was calculated.

In vivo cytotoxicity of light-inducible CAR T cells with in vitro light activation. NSG mice were subcutaneously injected with 1×10⁵ Fluc+Nalm-6 tumor cells on the shaved right flank, and randomly assigned to three groups: Light, Dark, and UIF (uninfected). Three days after tumor inoculation, primary human T cells infected with WW-LINTAD, light-inducible Cre (LexA-Pₘᵢₙᵢ-Cre) and loxP-CD19CAR (PGK-loxP-ZsGreen-STOP-loxP- CD19CAR) were divided into two groups, one receiving blue light stimulation at 1 s/30 s for 12 hr, with the other group kept in dark. One day after in vitro light/dark treatment (four days after tumor inoculation), these two groups of cells were locally injected into the mice in the Light and Dark groups, respectively, at the tumor sites (1×10⁶ cells/injection). Mice in the UIF group received local injection of 1×10⁶ uninfected primary human T cells. BLI was performed twice every week as described above. The integrated Fluc luminescence in a defined ROI was used to quantify tumor burden.

In vivo cytotoxicity of light-inducible CAR T cells with in vivo light activation. NSG mice were subcutaneously injected with 2×10⁵ Fluc+Nalm-6 cells on the left and right flanks, respectively, to generate two tumors. Four days after tumor inoculation, 6×10⁵ primary human T cells infected with WW-LINTAD, light-inducible Cre and LoxP-CD19CAR or uninfected primary human T cells were locally injected into both tumor sites. One flank was randomly chosen to be the side receiving blue light stimulation (Light), with the other side being the dark control (Dark). Four hours after local T cell injection, the light side was stimulated using the light box at 1 s/30 s for 12 hr, while the Dark side was covered by a piece of aluminum foil as described above. BLI was performed twice every week as described above to determine tumor aggressiveness.

Statistical analysis. Two-tailed Student's t-test with Bonferroni post-hoc correction was applied for the statistical analysis in FIG. 3a, FIG. 4a, b, and FIG. S2b. One-way ANOVA with Fisher's Least Significant Difference post-hoc test was applied for the statistical analysis in FIG. 4g, FIG. 5b, c, f, and FIG. 6c, d. Two-tailed Student's t-test was applied for the rest of the statistical analysis. *: P<0.05; **: P<0.01; ***: P<0.001.

### Figure Legend

FIG. 1. Schematics of the light-inducible nuclear translocation and dimerization (LINTAD) gene activation system. (a) The three components of the LINTAD system. LexA-CIB1-biLINuS (LCB): LexA fused with CIB1 and the bipartite light-inducible nuclear localization signal (biLINuS); CRY2PHR-VPR (CV): CRY2PHR fused with VPR; Light-inducible reporter: LexA binding sequence (LexA BS) fused with a minimal promoter and a target gene. In the dark state, LCB stays in the cytoplasm and CV in the nucleus. (b) Upon blue light stimulation, the Jα helix of LOV2 domain in biLINuS is unfolded to expose the NLS peptide (upper part in b), which leads to LCB translocation into the nucleus (red dashed line 1 in a). LexA then binds to LexA BS on the light-inducible reporter (red dashed line 2 in a). Meanwhile, CRY2PHR binds to CIB1 upon blue light stimulation (red dashed line 3 in a), thus targeting VPR to the minimal promoter region to trigger the reporter gene expression.

FIG. 2. Characterization of LINTAD gene activation in HEK 293T cells. (a) HEK 293T cells were transfected with LexA-CIB1-mCherry-biLINuS (LCmB) to track the nuclear localization of LCB before and after light stimulation. Scale bar, 20 μm. (b) Light-inducible mNeonGreen expression. HEK 293T cells were co-transfected with LCB, CV and the light-inducible mNeonGreen reporter. Dark, without light stimulation. Light, with 24 hr light stimulation. Scale bar, 200 μm. (c) Comparison of mNeonGreen (mNG) intensity of cells transfected with LINTAD and mNeonGreen reporter with (Light) or without light stimulation (Dark). NT, HEK 293T without transfection. AU, arbitrary unit.

Results were quantified by flow cytometry (n=4 independent experiments, with 20,000 cells per experiment). (d) Comparison of light-inducible systems with or without the light-inducible nuclear localization signal (biLINuS). HEK 293T cells were co-transfected with LexA-CIB1-biLINuS (With biLINuS) or LexA-CM1 (No biLINuS), CV, the light-inducible firefly luciferase reporter, and a constitutive Renilla luciferase as an internal reference to normalize the induced firefly luciferase expression in each group (n=3 independent experiments). Light induction fold in each group is defined as the ratio of the normalized Fluc activities in the light condition to that in the dark condition. (e) Comparison of CD47 binding of cells transfected with LCB, CV and the light-inducible CV1 reporter with or without light stimulation. Left: schematics of CV1-CD47 binding assay. Right: representative flow cytometry histograms of PE staining (Streptavidin-PE) of CD47 under different conditions. Dark, without light stimulation. Light, with 24 hr light stimulation. No CD47, cells without CD47 ligand incubation before PE staining. With CD47, cells incubated with CD47 before PE staining. **: P<0.01; ****: P<0.0001; two-tailed Student's t-test. Error bar: SEM.

FIG. 3. LINTAD system can induce target gene expression upon light stimulation in Jurkat cells. (a) Characterization of LINTAD system with light-inducible firefly luciferase (Fluc) reporter. Jurkat cells were transfected with (i) both LCB and CV components (LINTAD) or (ii) only the LCB component (LCB only) or (iii) only the CV component (CV only). All groups were also co-transfected with the light-inducible Fluc reporter. Fluc activities were measured using the same number of cells in each group, and all values were normalized to that of “CV only (dark)”. n=3 independent experiments. (b) Representative flow cytometry charts showing light-inducible CD19CAR expression in Jurkat cells (transfected with LINTAD regulators LCB and CV, and the light-inducible CD19CAR reporter; the whole live cell population is shown in each chart). CAR expression was quantified by staining of the Myc tag fused to the extracellular domain of CD19CAR. The gating threshold for CAR+cells was based on the Myc tag staining of non-transfected Jurkat cells, and was indicated in the figure with dotted line. (c) Comparison of CAR+cell percentage of the Dark and Light groups shown in b. n=3 independent experiments. (d) Representative flow cytometry charts showing CD69 levels of the Light and Dark groups. Jurkat cells were transfected with LINTAD regulators and the CD19CAR-YPet reporter, co-cultured with CD19-expressing Toledo cells after light/dark treatment, and stained with anti-CD69 antibody for flow cytometry analysis. The gating threshold for CD69+ cells was based on the staining of non-transfected Jurkat cells, and was indicated in the figure with dotted line. The YPet+populations of the co-cultured cells were used for CD69 comparison to exclude Toledo cells and Jurkat cells not containing all the three plasmids. See FIG. S3d for the CAR-YPet expression profiles of the Light and Dark groups. (e) Comparison of CD69+ cell percentage of the Dark and Light groups shown in d. n=3 independent experiments. (f) Representative CD69 expression profiles of Jurkat cells hosting light-inducible headless CAR reporter after co-culture with CD19-expressing target cells and anti-CD69 antibody staining. The YPet+populations were used for CD69 comparison. The gating threshold for CD69+ cells was based on the staining of non-transfected Jurkat cells, and was indicated in the figure with dotted line. *: P<0.05; **: P<0.01. Ns, not significant. Two-tailed Student's t-test with Bonferroni correction was used for panel a; two-tailed Student's t-test was used for panels c and e. In all panels: Light, with 24 hr light stimulation. Dark, without light stimulation. In all bar graphs: data represent mean values±SEM.

FIG. 4. Light-inducible cytotoxicity of engineered primary human T cells in vitro and light-inducible gene activation in vivo. (a) Characterization of LINTAD-mediated Fluc induction in primary human T cells. T cells infected with LINTAD regulators and Fluc reporter (with constitutive Rluc as internal reference) were (i) kept in dark (Dark) or stimulated by blue light for (ii) 6 hr, (iii) 12 hr, (iv) 24 hr. The gene induction level was represented by the ratio of Fluc to Rluc luminescence in each group, and normalized to the mean value of the Dark group. n=4 independent experiments. (b) In vitro light-inducible cytotoxicity of T cells with CD19CAR reporter. Engineered T cells were either without light stimulation (Dark) or stimulated with blue light for 6 hr or 12 hr, followed by co-culture with Fluc+ Nalm-6 tumor cells for 24 hr. Uninfected T cells (UIF) were used as control following the same procedures. The Fluc activities of the remaining live Nalm-6 cells after co-culture were quantified, and the inverses of the Fluc readings were calculated as the cytotoxicity values and normalized to that of the Dark group. n=3 independent experiments. (c-d) Cytokines IL-2 and IFN-y secretion from T cells engineered with LINTAD and CD19CAR reporter after co-culture with Nalm-6 cells quantified by ELISA. Dark, without light stimulation. Light, with 12 hr blue light stimulation. N>=3 biological repeats. (e-g) Light-inducible Fluc activation in vivo. (e) Mouse model used for in vivo light-inducible gene activation. Engineered light-inducible

Nalm-6 cells infected with LINTAD and Fluc reporter (with constitutive Rluc as internal reference) were subcutaneously injected into both flanks of NSG mice. One side of the mouse received blue light stimulation (Light Side) while the other side served as the dark control (Dark Side) as described in Materials and Methods. (f) Representative bioluminescence images showing the induced Fluc expression by blue light (at the region highlighted by the arrow). (g) Statistical comparison of light-inducible Fluc activation in mice. Induction level was indicated by Fluc/Rluc ratio at the same site. All Fluc/Rluc values were normalized to that of “Before, Dark Side”. n=5 mice. *: P<0.05; **: P<0.01; ***: P<0.001. Two-tailed Student's t-test with Bonferroni correction was applied in a and b. Two-tailed Student's t-test was applied in c and d. One-way Analysis of Variance (ANOVA) with Fisher's Least Significant Difference multiple comparison test was applied for g. Bar graphs: data represent mean values±SEM.

FIG. 5. In vivo cytotoxicity of light-activated primary human T cells. (a) Gene induction efficiency of LINTAD strongly depends on concentrations of regulators. Primary human T cells were infected with LCB-2A-tBFP and eGFP-2A-CV to indicate the expression levels of LINTAD regulators. Light-inducible mCherry reporter was used to determine the gene induction level. The intensity (and hence induction level) of mCherry is color coded, with cold and hot colors representing low and high levels. Dark, without light stimulation. Light, with 12 hr blue light stimulation. (b) Light-inducible gene expression using WW-LINTAD in primary T cells. Light and Dark: T cells infected with lentiviral WW-LINTAD (LWWCB and WP1CV) and Fluc reporter (with constitutive Rluc as internal reference) were stimulated with (Light) or without (Dark) for 12 hr. CTL: control T cells infected with only the LWWCB component and reporter. Fluc/Rluc ratios represent gene induction levels and are normalized to that of the Dark group. (c) Cytotoxicity of T cells engineered with WW-LINTAD, light-inducible Cre reporter and loxP-ZsGreen-stop-loxP-CD19CAR (see FIG. S9). UIF: uninfected T cells. CTL: control T cells infected with only the LWWCB component of WW-LINTAD, Cre reporter, and loxP-ZsGreen-stop-loxP-CD19CAR. Dark: without light stimulation. Light: with 12 hr light stimulation. Different groups of T cells were co-cultured with Fluc+Nalm-6 cells for 24 hr. Fluc activities of the remaining live Nalm-6 cells after co-culture were quantified. Cytotoxicity values were calculated as 1/Fluc and normalized to that of the Dark group. Data represent mean values±SEM (n=3 independent experiments). (d-f) In vivo cytotoxicity of light-inducible T cells after in vitro light stimulation. NSG mice were subcutaneously injected with Fluc+Nalm-6 cells on the right flank. Primary human T cells engineered with WW-LINTAD, light-inducible Cre reporter and loxP-ZsGreen-stop-loxP-CD19CAR were treated with (Light) or without (Dark) 12 hr blue light, followed by local injection into the mice at the tumor sites. Uninfected T cells (“UIF”) were used as control following the same procedures. (d) Timeline of tumor inoculation and T cell injection. (e) Tumor burden was quantified by bioluminescence imaging (BLI) after tumor inoculation for 16 days. We only included four mice in the Light group because the engineered T cells were not sufficient for five mice as planned due to accidental cell loss during experimental processes. (I) Quantification of tumor aggressiveness in different groups in e. The integrated luminescence of a tumor at each time point was normalized to that of the same tumor on Day 3 (before T cell injection). Data represent mean values±SEM (n=4 mice for “Light”, n=5 mice for “Dark” and “UIF”). *: P<0.05; **: P<0.01; ***: P<0.001. One-way ANOVA followed by Fisher's Least Significant Difference multiple comparison test was used for panels b, c, and f).

FIG. 6. In vivo light stimulation of LINTAD CAR T cells can control cytotoxicity with high spatial resolution. (a) Mouse model and experiment timeline. Fluc+Nalm-6 cells were subcutaneously injected on both flanks of the mouse, and LINTAD CAR T cells were locally injected into the inoculated tumor sites 4 days later. One side (left) was illuminated with blue light for 12 hr (Light) while the other side (right) served as the dark control (Dark) as described in Materials and Methods. (b) Bioluminescence images of mice in the experiment described in a. (c) Quantification of tumor growth in b. (d) Same experiment procedures as in a with uninfected T cells instead of LINTAD CAR T cells.

In all panels: tumor size was quantified using the integrated Fluc luminescence of a tumor normalized to that of the same tumor on Day 3. Error bar, SEM (n=10 mice in b and n=5 mice in c and d). *: P<0.05; **: P<0.01; ***: P<0.001. One-way ANOVA followed by Fisher's Least Significant Difference multiple comparison test was used for panels c and d.

FIG. S1. Nuclear translocation of LexA-CIB1-mCherry-biLINuS is specific to blue light stimulation. (a) Fluorescence images of HEK 293T cells expressing LexA-CIB1-mCherry-biLINuS (LCmB) before and after 10 min of 560 nm light stimulation (0.5 s/ 30 s). Scale bar, 20 μm. (b) Fluorescence images of the same cells in a before and after 10 min of 460 nm light exposure (0.5 s/ 30 s). Scale bar, 20 μm. (c) A representative image showing the nuclear localization of EGFP-CV in HEK 293T cells. Scale bar, 20 μm. (d) Fluorescence images of HEK 293T cells expressing eGFP with (Light) or without (Dark) 24 hr light stimulation. Scale bar, 200 μm. (e) Fluorescence images of HEK 293T cells expressing tBFP with (Light) or without (Dark) 24 hr light stimulation. Scale bar, 200 μm. Photobleaching was observed in both cases.

FIG. S2. Characterization of LINTAD-mediated gene induction in HEK 293T cells using dual-luciferase reporter system. (a) DNA cassettes used in the dual-luciferase reporter system. LexA BS: LexA-binding DNA sequence. Fluc, firefly luciferase gene. TK, constitutive HSV-thymidine kinase promoter. Rluc, Renilla luciferase gene. Stop codon after each gene is indicated. (b) Light-inducible gene activation using LINTAD in HEK 293T cells. Cells were transfected with intact LINTAD system (LCB and CV, shown as “LINTAD” in figure) or LCB (“LCB only”) or CV (“CV only”), together with the dual-luciferase reporter constructs shown in a. Twenty-four hr after transfection, cells were stimulated with blue light for 24 hr (1s/30s, “Light”) or kept in dark for 24 hr (“Dark”), n=3 independent experiments. Ns, not significant; *: P<0.05; **: P<0.01; ***: P<0.001. Two-tailed Student's t-test with Bonferroni correction. Error bar: SEM. (c) Comparison of LINTAD systems using CRY2PHR and CRY2low in HEK 293T cells. For the original LINTAD group, cells were co-transfected with LCB and CV-expressing plasmids. For the LINTAD (CRY2low) group, cells were transfected with LCB and CRY2low-VPR. Both groups were also transfected with the light-inducible firefly luciferase reporter and the constitutive Renilla luciferase-expressing plasmid as internal control. Data represent mean values±standard deviation (n=3 experiments). ****P<0.0001; two-tailed Student's t-test.

FIG. S3: LINTAD-mediated light-inducible gene expression in Jurkat cells. Jurkat cells were transfected with LINTAD system (LCB and CV) with different light-inducible reporter cassettes (mNeonGreen reporter in a and c; CD19CAR-YPet in b and d). Cells were stimulated with blue light for 24 hr (Light) or kept in dark for 24 hr (Dark) before fluorescence measurement. (a-b) Comparison of mean fluorescent intensity, n=3 independent experiments, 10,000 cells in each experiment. Two-tailed Student's t-test. *, P<0.05; **, P<0.01; ***, P<0.001. Error bars, SEM. (c-d) Representative fluorescence profiles of light-induced or non-induced cells shown in a orb. The YPet+gate was indicated with dotted line in d. (e) Dynamics of CD19CAR expression after light induction in Jurkat cells. Light stimulation was applied from day 1 to day 2 for 24 hr as indicated by the bar below the x-axis. CD19CAR expression was measured by myc-tag staining. (f) Representative expression (YPet fluorescence) profiles of Jurkat cells transfected with LCB, CV and the light-inducible YPet-tagged headless CAR (without CD19 recognition domain) reporter with (Light) or without (Dark) 24 hr light stimulation.

FIG. S4: Lentiviral vectors used for T cell infection. (a) The constitutive LCB lentiviral vector contains PGK promoter followed by LCB gene, WPRE element, flanked by 5′LTR and 3′LTR. (b) The constitutive CV viral vector has the same design as LCB lentiviral vector, except containing the CV gene instead of LCB. (c) The light-inducible Fluc reporter lentiviral vector contains the LexA binding sequence (LexA BS), followed by minimal promoter, Fluc gene, PGK promoter, and Rluc gene (for Fluc normalization). (d)

The light-inducible CD19CAR reporter lentiviral vector contains LexA BS, minimal promoter and Myc-tagged CD19CAR gene and WPRE element, flanked by 5′LTR and 3′LTR. (e) Infection efficiency of LINTAD system in primary T cells. Primary T cells were co-infected with lentiviruses encoding LCB-P2A-tBFP, EGFP-P2A-CV, and LIP-myc-CD19CAR-PGK-mCherry. Left, the mCherry expression profile in the infected T cells. Right, the EGFP-tBFP expression profile of mCherry+cells.

FIG. S5: Light stimulation system used for in vivo studies. a, overview of the light stimulation system containing home-built LED control box with intensity knobs (for two LEDs), on/off timer (for controlling on/off cycle pattern of LEDs), and blue-light emitting LED. Each item was indicated in the figure. b, a representative mouse with a blue LED attached on the desired stimulation region. c, Home-built LED control box with intensity knobs. The blue LED used in b is also shown here (wavelength 460 nm, diameter 3 mm).

FIG. S6: The level of LINTAD gene activation depends on the expression level of regulators. (a-b) HEK 293T cells transfected with eGFP-2A-CV, and LCB-2A-tBFP and CD19CAR reporter cassette were stimulated with light (“Light”) or kept in dark (“Dark”) for 24 hr before flow cytometry measurement. Induced CAR expression were measured by anti-CAR antibody staining (goat anti-mouse IgG F(ab′)2 fragment, Jackson Immuno-Research Laboratories, #115-606-072). A threshold on eGFP-2A-CV expression for efficient CAR induction can be clearly seen from the flow cytometry chart. (c) Three-dimensional plots showing the relationship between gene induction and regulator expression levels. Human primary T cells were infected with viral vectors expressing LCB-2A-tBFP and eGFP-2A-CV (or eGFP-2A-CVP64 using VP64 instead of VPR as the transcriptional activator as indicated in figure) and the viral vector of light-inducible mCherry reporter. Number on axes indicate relative expression levels of each regulator component. mCherry expression level is represented by color, with cold and hot colors representing low and high levels, respectively.

FIG. S7: Engineering of LINTAD with weak dimer helpers. (a) Construct designs of LINTAD with various weak dimer helpers. LINTAD, original LINTAD system without weak dimer helper. G1-10, split super folder GFP1-10 fragment. G11, split super folder GFP11 fragment. Number (−1 or −2) after name of each group indicates different designs of the same weak dimer pair. (b) Comparison of light-induction capability of the above systems at different regulator expression levels. HEK 293T cells cultured in 24-well plates were transfected with different amounts of regulator-expressing constructs (DNA amounts were indicated at X-axis) and dual-luciferase reporter cassettes (same amount for all the groups). Cells were stimulated with blue light (Light) or kept in dark (Dark) for 24 hr before luciferase measurement. “Fold change” represents the ratio of (Fluc/Rluc)Light/(Fluc/Rluc)Dark, to compare the light induction capability of each design, where Fluc and Rluc represent the relative luciferase activities of firefly luciferase and Renilla luciferase, respectively.

FIG. S8: LINTAD with weak dimer helpers in T cells. Primary human T cells were infected with viral vectors expressing WW-2 (indicated as “WW” in figure) or SH3-2 (indicated as “SH3” in figure, see FIG. S7a) and viral vector of dual-luciferase reporter. Cells were stimulated with light (12 hr or 24 hr) or kept in dark (Dark) before measurement. Reporter, cells infected with reporter viral vector only. Error bar: standard deviation. *: P<0.05; **: P<0.01; ***: P<0.001. Error bars, SEM. Two-tailed Student's t-test. (b-c) In vivo light stimulation of LINTAD CART cells can control cytotoxicity with high spatial resolution (plots showing individual data points for FIG. 6c-d). (b) LINTAD CAR T cells were subcutaneously injected into two sites of the same mice (left and right flanks), where tumor cells (Nalm-6 with constitutive Fluc) had been inoculated 4 days before. Left side was illuminated with blue light for 12 hr (light). Right side was covered with foil (dark). Tumor size was measured by Fluc luminescence reading. All luminescence values were normalized to that of the same tumor on day 3 after tumor inoculation. (c) Same experiment procedures as in b, but uninfected T cells were used instead of LINTAD CAR T cells. Tumor size was measured by Fluc luminescence reading. All luminescence values were normalized to that of the same tumor on day 3 after tumor inoculation. Error bar, SEM (n=5 mice in each group). Two-tailed Student's t test. Error bar, SEM (n=5 mice in each group). Two-tailed Student's t test. *: P<0.05; **: P<0.01; ***: P<0.001.

FIG. S9: Design and lentiviral vectors used for in vivo cytotoxicity studies. (a) Schematics of light-inducible Cre reporter and loxP-ZsGreen-STOP-loxP-CD19CAR cassettes in the cells (LINTAD regulators not shown). Once LINTAD system in the cell is activated by light, Cre can be induced to express and catalyze recombination between loxP sites on the loxP-ZsGreen-STOP-loxP-CD19CAR cassette, thus switching on constitutive CD19CAR expression in the cells. (b) Lentiviral vectors for expression of WW-LINTAD (WW-2 in FIG. S7a) and light-inducible Cre and loxP-ZsGreen-STOP-loxP-CD19CAR (before and after recombination) cassette. All viral vectors mentioned above are shown.

### REFERENCES

1. M. V. Maus, C. H. June, Making Better Chimeric Antigen Receptors for Adoptive T-cell Therapy. Clin Cancer Res 22, 1875-1884 (2016).
2. E. Jacoby et al., CD19 CAR immune pressure induces B-precursor acute lymphoblastic leukaemia lineage switch exposing inherent leukaemic plasticity. Nat Commun 7, 12320 (2016).
3. C. Y. Wu, K. T. Roybal, E. M. Puchner, J. Onuffer, W. A. Lim, Remote control of therapeutic T cells through a small molecule-gated chimeric receptor. Science 350, aab4077 (2015).
4. M. J. Frigault et al., Identification of Chimeric Antigen Receptors That Mediate Constitutive or Inducible Proliferation of T Cells. Cancer Immunology Research, (2015).
5. N. Tokarew, J. Ogonek, S. Endres, M. von Bergwelt-Baildon, S. Kobold, Teaching an old dog new tricks: next-generation CAR T cells. Br J Cancer 120, 26-37 (2019).
6. F. Caliendo, M. Dukhinova, V. Siciliano, Engineered Cell-Based Therapeutics: Synthetic Biology Meets Immunology. Front Bioeng Biotechnol 7, 43 (2019).
7. J. Fisher et al., Avoidance of On-Target Off-Tumor Activation Using a Co-stimulation-Only Chimeric Antigen Receptor. Mol Ther 25, 1234-1247 (2017).
8. G. Gross, Z. Eshhar, Therapeutic Potential of T Cell Chimeric Antigen Receptors (CARs) in Cancer Treatment: Counteracting Off-Tumor Toxicities for Safe CAR T Cell Therapy. Annu Rev Pharmacol Toxicol 56, 59-83 (2016).
9. R. A. Morgan et al., Case Report of a Serious Adverse Event Following the

Administration of T Cells Transduced With a Chimeric Antigen Receptor Recognizing ERBB2. Molecular Therapy 18, 843-851 (2010).
10. C. A. Ramos, B. Savoldo, G. Dotti, CD19-CAR trials. Cancer J20, 112-118 (2014).
11. C. R. Cruz et al., Adverse events following infusion of T cells for adoptive immunotherapy: a 10-year experience. Cytotherapy 12, 743-749 (2010).
12. J. Smith, J. Valton, A universal suicide switch for chimeric antigen receptor T cell adoptive therapies. Journal of Clinical Oncology 34, 7039-7039 (2016).
13. M. J. Brenner, J. H. Cho, N. M. L. Wong, W. W. Wong, Synthetic Biology: Immunotherapy by Design. Annu Rev Biomed Eng 20, 95-118 (2018).
14. V. D. Fedorov, M. Themeli, M. Sadelain, PD-1- and CTLA-4-based inhibitory chimeric antigen receptors (iCARs) divert off-target immunotherapy responses. Science translational medicine 5, 215ra172 (2013).
15. J. H. Cho, J. J. Collins, W. W. Wong, Universal Chimeric Antigen Receptors for Multiplexed and Logical Control of T Cell Responses. Cell 173, 1426-1438 e1411 (2018).
16. K. T. Roybal et al., Precision Tumor Recognition by T Cells With Combinatorial Antigen-Sensing Circuits.Cell 164, 770-779 (2016).
17. D. Niopek et al., Engineering light-inducible nuclear localization signals for precise spatiotemporal control of protein dynamics in living cells. Nat Commun 5, 4404 (2014).
18. M. J. Kennedy et al., Rapid blue-light-mediated induction of protein interactions in living cells. Nat Methods 7, 973-975 (2010).
19. S. Konermann et al., Optical control of mammalian endogenous transcription and epigenetic states. Nature 500, 472-476 (2013).
20. K. Muller et al., A red/far-red light-responsive bi-stable toggle switch to control gene expression in mammalian cells. Nucleic Acids Res 41, e77 (2013).
21. H. Yumerefendi et al., Light-induced nuclear export reveals rapid dynamics of epigenetic modifications. Nat Chem Biol 12, 399-401 (2016).
22. A. Levskaya, O. D. Weiner, W. A. Lim, C. A. Voigt, Spatiotemporal control of cell signalling using a light-switchable protein interaction. Nature 461, 997-1001 (2009).
23. P. Kyriakakis et al., Biosynthesis of Orthogonal Molecules Using Ferredoxin and Ferredoxin-NADP(+) Reductase Systems Enables Genetically Encoded PhyB Optogenetics. ACS Synth Biol 7, 706-717 (2018).
24. A. A. Kaberniuk, A. A. Shemetov, V. V. Verkhusha, A bacterial phytochrome-based optogenetic system controllable with near-infrared light. Nat Methods 13, 591-597 (2016).
25. A. Taslimi et al., Optimized second-generation CRY2-CIB dimerizers and photoactivatable Cre recombinase. Nat Chem Biol 12, 425-430 (2016).
26. L. R. Polstein, C. A. Gersbach, A light-inducible CRISPR-Cas9 system for control of endogenous gene activation. Nat Chem Biol 11, 198-200 (2015).
27. A. Chavez et al., Highly efficient Cas9-mediated transcriptional programming. Nat Methods 12, 326-328 (2015).
28. H. H. Chen, H. I. Yu, M. H. Yang, W. Y. Tarn, DDX3 Activates CBC-eIF3-Mediated Translation of uORF-Containing Oncogenic mRNAs to Promote Metastasis in HNSCC. Cancer Res 78, 4512-4523 (2018).
29. K. Weiskopf et al., Engineered SIRPalpha variants as immunotherapeutic adjuvants to anticancer antibodies. Science 341, 88-91 (2013).
30. J. Seong et al., Detection of focal adhesion kinase activation at membrane microdomains by fluorescence resonance energy transfer. Nat Commun 2, 406 (2011).
31. D. L. Che, L. Duan, K. Zhang, B. Cui, The Dual Characteristics of Light-Induced Cryptochrome 2, Homo-oligomerization and Heterodimerization, for Optogenetic Manipulation in Mammalian Cells. ACS Synth Biol 4, 1124-1135 (2015).
32. D. Cibrian et al., CD69 controls the uptake of L-tryptophan through LAT1-CD98 and AhR-dependent secretion of IL-22 in psoriasis. Nat Immunol 17, 985-996 (2016).
33. S. Cabantous, T. C. Terwilliger, G. S. Waldo, Protein tagging and detection with engineered self-assembling fragments of green fluorescent protein. Nat Biotechnol 23, 102-107 (2005).
34. K. E. Miller, Y. Kim, W. K. Huh, H. O. Park, Bimolecular Fluorescence Complementation (BiFC) Analysis: Advances and Recent Applications for Genome-Wide Interaction Studies. J Mol Biol 427, 2039-2055 (2015).
35. R. Grunberg et al., Engineering of weak helper interactions for high-efficiency FRET probes. Nat Methods 10, 1021-1027 (2013).
36. K. Muller et al., Synthesis of phycocyanobilin in mammalian cells. Chem Commun (Camb) 49, 8970-8972 (2013).
37. C. Ash, M. Dubec, K. Donne, T. Bashford, Effect of wavelength and beam width on penetration in light-tissue interaction using computational methods. Lasers Med Sci 32, 1909-1918 (2017).
38. S. Chen et al., Near-infrared deep brain stimulation via upconversion nanoparticle-mediated optogenetics. Science 359, 679-684 (2018).
39. S. Il Park et al., Soft, stretchable, fully implantable miniaturized optoelectronic systems for wireless optogenetics. Nat Biotechnol 33, 1280-+(2015).
40. J. W. Jeong et al., Wireless Optofluidic Systems for Programmable In Vivo Pharmacology and Optogenetics. Cell 162, 662-674 (2015).
41. R. G. Fox et al., Image-based detection and targeting of therapy resistance in pancreatic adenocarcinoma. Nature 534, 407-411 (2016).
42. J. Lemke, S. von Karstedt, J. Zinngrebe, H. Walczak, Getting TRAIL back on track for cancer therapy. Cell Death Differ 21, 1350-1364 (2014).
43. C. Ede, X. Chen, M. Y. Lin, Y. Y. Chen, Quantitative Analyses of Core Promoters Enable Precise Engineering of Regulated Gene Expression in Mammalian Cells. ACS Synth Biol 5, 395-404 (2016).
44. X. J. Gao, L. S. Chong, M. S. Kim, M. B. Elowitz, Programmable protein circuits in living cells. Science 361, 1252-1258 (2018).
45. K. T. Roybal et al., Engineering T Cells with Customized Therapeutic Response Programs Using Synthetic Notch Receptors. Cell 167, 419-432 e416 (2016).
46. M. Boice et al., Loss of the HVEM Tumor Suppressor in Lymphoma and Restoration by Modified CAR-T Cells. Cell 167, 405-418 e413 (2016).
47. L. Duan et al., Understanding CRY2 interactions for optical control of intracellular signaling. Nat Commun 8, 547 (2017).
48. J. Eyquem et al., Targeting a CAR to the TRAC locus with CRISPR/Cas9 enhances tumour rejection. Nature 543, 113-117 (2017).
49. J. Sun et al., Engineered proteins with sensing and activating modules for automated reprogramming of cellular functions. Nat Commun 8, 477 (2017).

A number of embodiments of the invention have been described. Nevertheless, it can be understood that various modifications may be made without departing from the spirit and scope of the invention. Accordingly, other embodiments are within the scope of the following claims.

1. A method for remotely-controlling and non-invasively manipulating expression of an exogenous nucleic acid in a cell, or an immune cell,
the method comprising:
(a) inserting or expressing in a recombinantly engineered cell: a blue-light-mediated light-inducible nuclear translocation and dimerization (LINTAD) system comprising:
(i) a LexA-CIB1-biLINuS or LCB cassette or chimeric nucleic acid comprising: a LexA DNA-binding domain fused to an N-terminus of CIB1 fused to a biLINuS cassette or chimeric nucleic acid comprising a LOV2 domain of an Jα helix and a bipartite light-inducible nuclear localization signal (NLS), wherein the biLINuS is fused to the C-terminus or CIB1,
(ii) a CRY2-VPR or CV cassette or chimeric nucleic acid comprising: an nuclear location signal peptide (NLS) fused to an N-terminus of CRY2PHR (photolyase homology region of Arabidopsis CRY2, amino acids 1-498) and a strong transcription activator VPR fused to the CRY2PHR C-terminus, and
(iii) a cassette or chimeric nucleic acid comprising: the exogenous nucleic acid operatively linked to a promoter, operatively linked to a LexA binding site (BS), wherein the exogenous nucleic acid is expressed when the LexA BS binds to LexA;
(b) exposing the cell to a blue light to drive to activate biLINuS in LCB, thereby unfolding the Jα helix of LOV2 domain and exposing its NLS motif to cause the nuclear translocation of LCB; simultaneously, the CRY2PHR domain in CV is also activated by blue light and can bind the CIB1 domain of LCB with high affinity, and the LCB-CV complex is targeted to the LexA BS on the reporter cassette so that VPR is in close proximity to the promoter, triggering transcription of the exogenous nucleic acid.

2. The method of claim 1, wherein the recombinantly engineered cell is administered in vivo, optionally the recombinantly engineered cell is administered to an individual in need thereof in vivo, and optionally the blue light is administered to only a desired area or location in the individual in need thereof, and optionally the desired area or location in the individual in need thereof is a site of a tumor or a growth, and optionally the recombinantly engineered cell is injected into and/or adjacent or approximate to a cancer of a site of a tumor or a growth.

3. The method of claim 1, wherein the expressing of the floxed exogenous nucleic acid in the cell adds a function to the cell, or immune cell, or manipulates a physiologic and/or a genetic process in the cell, or immune cell, and optionally when the upregulated nucleic acid is a nucleic acid expressing (encoding) a CAR, a single chain antibody, or a single-domain antibody (also known as sdAb or nanobody) or an antibody fragment consisting of a single monomeric variable antibody domain, or a regulator of CRISPR (clustered regularly interspaced short palindromic repeats), such as Cas9 and dCas9, to control endogenous genome and epigenome regulations, and genome editing, thereby adding a new specificity, function or target cell to a cell, an immune cell or a T cell.

4. The method claim 1, wherein the cell is a human cell or a mammalian cell, or is a recombinantly engineered cell engineered to be transplanted or inserted into a tissue, an organ, an organism or an individual, or is or comprises a non-human transgenic animal genetically engineered to contain one or a plurality of recombinantly engineered cells.

5. The method claim 1, wherein the cell or the individual in need thereof is first exposed to or administered tamoxifen followed by being exposed to or administered a continuous or pulsed blue light,
wherein optionally the cells are exposed to between about 400 to 600 nM 4-OHT, or about 500 nM 4-OHT, and optionally blue light is applied to the cells between about 2 to 5 hours, or about 3 hours, and optionally the blue light frequency is about 400 to 500 nM, and optionally the blue light is applied in a pulsed manner at about 1 second on to about 59 seconds off, or at about 5 seconds on to about 55 seconds off, optionally repeated over a time period of between about 1 hours and 36 hours, or between about 12 hours and 24 hours, and optionally the blue light is continuously applied to the cells for between about 1 hour and 24 hours, or between about 2 hours and 12 hours.

6. The method claim 1, wherein a chimeric antigen receptor (CAR) is expressed on a T cell surface after exposure of the T cell to tamoxifen followed by blue light, thereby activating the T cell to attack and/or kill a cancerous tissue, a cancer cell or a tumor cell,
wherein optionally the cancerous tissue, cancer cell or tumor cell is a local or skin or mucosal metastatic head/neck cancer, a melanoma, or a skin cancer or a skin growth.

7. The method claim 1, wherein the cell is inside the body of an animal or a human in need thereof, and the recombinantly engineered cell is focused on or approximate to a tumor or a dysplastic or dysfunctional tissue.

8. The method claim 1, wherein the method is used for the manipulation or correction of a pathological process, optionally, for eradicating a tumor or a cancer in an individual in vivo, wherein optionally the individual is a human or an animal.

9-10. (canceled)

11. A genetically engineered cell as engineered for use in claim 1, for use as a medicament, or for use as a medicament in a remotely-controlled and non-invasive manipulation of a physiologic and/or a genetic process in a cell, or an immune cell, or for the addition of a function or a target specificity to the cell, or immune cell, or plurality of cells or immune cells, or for the manipulation or correction of a pathological process, optionally, for eradicating a tumor or a cancer in an individual in vivo,.
wherein the genetically engineered cell comprises or has contained therein:
a recombinantly engineered cell, wherein optionally the recombinantly engineered cell is an immune cell or comprises a plurality of cells or immune cells: a blue-light-mediated light-inducible nuclear translocation and dimerization (LINTAD) system comprising:
(i) a LexA-CIB1-biLINuS or LCB cassette or chimeric nucleic acid comprising: a LexA DNA-binding domain fused to an N-terminus of CIB1 fused to a biLINuS cassette or chimeric nucleic acid comprising a LOV2 domain of an Jα helix and a bipartite light-inducible nuclear localization signal (NLS), wherein the biLINuS is fused to the C-terminus or CIB1,
(ii) a CRY2-VPR or CV cassette or chimeric nucleic acid comprising: an nuclear location signal peptide (NLS) fused to an N-terminus of CRY2PHR (photolyase homology region of Arabidopsis CRY2, amino acids 1-498) and a strong transcription activator VPR fused to the CRY2PHR C-terminus, and
(iii) a cassette or chimeric nucleic acid comprising: the exogenous nucleic acid operatively linked to a promoter, optionally a minimal promoter, operatively linked to a LexA binding site (BS), wherein the exogenous nucleic acid is expressed when the LexA BS binds to LexA.

12. A kit or formulation comprising a genetically engineered cell of claim 11.

13. A synthetic or chimeric nucleic acid comprising a blue-light-mediated light-inducible nuclear translocation and dimerization (LINTAD) system comprising:
(i) a LexA-CIB1-biLINuS or LCB cassette or chimeric nucleic acid comprising: a LexA DNA-binding domain fused to an N-terminus of CIB1 fused to a biLINuS cassette or chimeric nucleic acid comprising a LOV2 domain of an Jα helix and a bipartite light-inducible nuclear localization signal (NLS), wherein the biLINuS is fused to the C-terminus or CIB1,
(ii) a CRY2-VPR or CV cassette or chimeric nucleic acid comprising: an nuclear location signal peptide (NLS) fused to an N-terminus of CRY2PHR (photolyase homology region of Arabidopsis CRY2, amino acids 1-498) and a strong transcription activator VPR fused to the CRY2PHR C-terminus, and
(iii) a cassette or chimeric nucleic acid comprising: the exogenous nucleic acid operatively linked to a promoter, optionally a minimal promoter, operatively linked to a LexA binding site (BS), wherein the exogenous nucleic acid is expressed when the LexA BS binds to LexA;
wherein optionally the blue-light-mediated LINTAD system is stably integrated into the genome of the cell, and optionally at least one component of the blue-light-mediated LINTAD system comprises SEQ ID NO:1 or SEQ ID NO:2.

14. A kit or formulation comprising a blue-light-mediated LINTAD system as set forth in claim 13.

15. An expression vehicle, a recombinantly engineered virus, or a vector comprising or having contained therein at least one component of a blue-light-mediated LINTAD system as set forth in claim 13, wherein optionally the recombinantly engineered virus is a lentivirus, and optionally the at least one component of a blue-light-mediated LINTAD system comprises SEQ ID NO:1 or SEQ ID NO:2.

16. The method of claim 1, wherein the immune cell is a T cell, a primary T cell, a B cell, a monocyte, a macrophage, a dendritic cell or a natural killer cell.

17. The method of claim 1, wherein the exogenous nucleic acid is contained in a vector or expression cassette.

18. The method of claim 1, wherein the exogenous nucleic acid comprises a nucleic acid encoding (expressing) a protein, and optionally the protein is a therapeutic protein, or a transcriptional or translational regulatory protein, or a receptor, or a recombinant or an artificial T cell receptor (also known as a chimeric T cell receptor, a chimeric immunoreceptor, a chimeric antigen receptor (CAR), an antibody, a single chain antibody, or a single-domain antibody (also known as sdAb or nanobody) or an antibody fragment consisting of a single monomeric variable antibody domain, or a regulator of CRISPR (clustered regularly interspaced short palindromic repeats), or Cas9 and dCas9, to control endogenous genome and epigenome regulations, and genome editing.

19. The method of claim 1, wherein the blue-light-mediated LINTAD system is stably integrated into the genome of the cell.

20. The method of claim 1, wherein the recombinantly engineered cell is an immune cell or comprises a plurality of cells or immune cells.

21. The method of claim 1, wherein the method comprises remotely-controlling and non-invasively manipulating expression of an exogenous nucleic acid in a cell, or an immune cell, and modifying or adding a target capability or a function to the cell, or immune cell.

22. The method of claim 1, wherein the cassette or chimeric nucleic acid comprises an exogenous nucleic acid operatively linked to a minimal promoter.


SEQUENCE LISTING (2 sequences)

SEQ ID NO: 1 - protein, 574 aa, artificial sequence
  synthetic polypeptide
MKALTARQQEVFDLIRDHISQTGMPPTRAEIAQRLGFRSPNAAEEHLKALARKGVIEIVSGASRGIRLLQEEEEGLPLVGRVAAGEPASGGGGSGGGGSNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIHVRARRGQATDSHSIAERVRREKISERMKFLQDLVPGCDKITGKAGMLDEIINYVQSLQRQIEFLSMKLAIVNPRPDFDMDDIFAKEVASTPMTVVPSPEMVLSGYSHEMVHSGYSSEMVNSGYLHVNPMQQVNTSSGGGGSGGGGSGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKRLPDANLAAAAKKKKLD

SEQ ID NO: 2 - protein, 1064 aa, artificial sequence
  synthetic polypeptide
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPASPKKKRKVEASSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF